(19) United States
(12) Patent Application Publication
(10) Pub. No.: US 2024/0316223 A1
(43) Pub. Date: Sep. 26, 2024

(54) Oxidative Cyclization Reagents for Chemoselective Tryptophan Bioconjugation

(71) Applicant: The Regents of the University of California, Oakland, CA (US)

(72) Inventors: Patrick J. Moon, Berkeley, CA (US); Xaio Xie, Berkeley, CA (US); F. Dean Toste, Berkeley, CA (US); Christopher J. Chang, Berkeley, CA (US)

(73) Assignee: The Regents of the University of California, Oakland, CA (US)

(21) Appl. No.: 18/731,289

(22) Filed: Jun. 1, 2024

Related U.S. Application Data
(63) Continuation of application No. PCT/US22/80201, filed on Nov. 19, 2022.
(60) Provisional application No. 63/291,349, filed on Dec. 18, 2021.

(51) Int. Cl. A61K 51/04; G01N 33/68

(52) U.S. Cl. CPC A61K 51/0406; G01N 33/6812; G01N 33/6848; G01N 2570/00

(57) ABSTRACT

A redox-based strategy for bioconjugation of tryptophan uses oxaziridine reagents that mimic oxidative cyclization reactions in indole-based alkaloid biosynthetic pathways to achieve highly selective and rapid tryptophan labeling.

### CROSS-REFERENCES TO RELATED APPLICATIONS

This application is a continuation of PCT/US22/80201, filed Nov. 19, 2022, which claims priority to U.S. Provisional Application No. 63/291,349, filed Dec. 18, 2021, the disclosures of which are hereby incorporated by reference in its entirety for all purposes.

### INTRODUCTION

Tryptophan (Trp) occupies a privileged place in biology owing to its versatile and unique chemistry (1,2). It is accounting for only 1% of amino acids observed in proteins compared to the expected 5% average amino acid frequency (2). As such, tryptophan is an attractive target for site-specific protein functionalization to enable diverse applications, including probes of protein function, activity-based proteomics for functional tryptophan identification and characterization, synthesis of covalent small-molecule inhibitors and activators, and antibody-drug conjugates (3-6). The distinct chemical reactivity of tryptophan arises from its indole moiety, which is hydrophobic and involved in a variety of bonding interactions (e.g., π-π, cation-π, and hydrogen bonds) (7,8). Compared to its low proteomic abundance, tryptophan is often enriched in functional sites in proteins, contributing to processes such as protein folding and signal transduction (1, 9-11). In this context, tryptophan residues are commonly thought of as buried within interior protein cores to hold/stabilize protein structures, but there is an increasing appreciation for the contributions of solvent-accessible surface tryptophan sites to protein function, including protein trafficking and protein-protein interactions (12,13). Indeed, post-translational modifications of surface-exposed tryptophans, such as oxidation and C-mannosylation, are critical nodes in signal transduction pathways. For example, irreversible oxidation of tryptophan by reactive oxygen species (ROS) can facilitate subsequent metal-binding and/or tryptophan dimerization processes resulting in loss of function (14-16). In contrast, tryptophan C-mannosylation, which occurs in a co-translational fashion in endoplasmic reticulum (ER) catalyzed by DPY19 enzymes, supports the formation of tryptophan-arginine ladders to enhance thermal stability and targeting to the secretory pathways (17,18).

In contrast to the myriad of acid-base approaches for functionalizing cysteine and lysine residues, which are highly nucleophilic, methods for tryptophan modification remain underdeveloped. Despite advances in this area, including transition metal-catalyzed C—H activation (19-22) and photochemically- and electrochemically-induced radical processes (23-26), reactions that enable site-selective tryptophan bioconjugation in proteins and proteomes and avoid harsh reaction conditions and/or side reactions are an unmet need (2,4). In particular, a major obstacle in developing an effective chemoselective tryptophan bioconjugation reaction under physiological conditions is its relatively weak nucleophilicity at neutral pH, precluding typical acid-base ligation processes. As such, we disclose a redox-based method, termed Tryptophan Chemical Ligation by Cyclization (Trp-CLiC), for selective and rapid tryptophan functionalization from peptides and proteins to proteomes.

### SUMMARY OF THE INVENTION

The invention provides methods and compositions for chemoselective cyclization conjugation to indole substrates, including oxidative cyclization reagents for chemoselective tryptophan bioconjugation, and methods of use.

In an aspect the invention provides a redox-based strategy for bioconjugation of tryptophan, the rarest amino acid, using oxaziridine reagents that mimic oxidative cyclization reactions in indole-based alkaloid biosynthetic pathways to achieve highly selective and rapid tryptophan labeling.

In embodiments the invention provides for appending payloads to solvent-accessible surface tryptophan residues on peptides and proteins, enabling functionalization of antibodies, detection of stress-induced protein unfolding, and profiling of hyperreactive tryptophan sites in whole proteomes.

In an aspect, the invention provides a synthetic method for selective and rapid tryptophan bioconjugation in proteins and proteomes by functional mimicry of oxidative cyclization processes in indole-based alkaloid biosynthesis.

In an aspect the invention provides a method of chemoselective conjugation comprising reacting an N-sulfonyl oxaziridine with an indole substrate in an oxidative cyclization reaction in an aqueous (preferably ≥90% or ≥95% water), biocompatible environment under conditions to form a resultant cycloadduct conjugation product.

The biocompatible environment is non-denaturing and generally compatible with the preservation of protein structure and function; and in particular, as applied to the subject proteins of the reaction. The conditions are distinct from reactions in generally denaturing organic solvents, with simple indoles, where many different chemical products are formed.

In embodiments the N-sulfonyl oxaziridine is of formula I, the indole substrate is of formula II, and the cycloadduct is of the corresponding formula III:

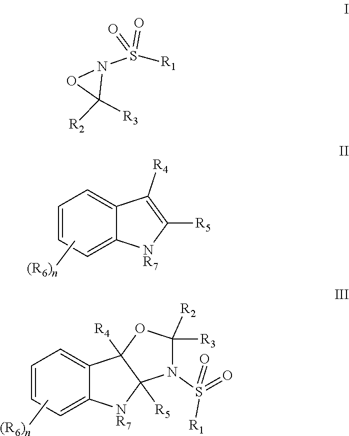

wherein R1-R7 are independently selected from optionally substituted heteroatom and optionally substituted, optionally hetero-, optionally cyclic C1-C18 hydrocarbyl, and n is an integer 1-5, preferably 1-3 or 1-2.

In embodiments:
R1-R3 and R5-R7 are independently H, C1-C4 alkyl (Me, Et, Pr, Bu) or fully or partially fluorinated C1-C4 alkyl (e.g. CF₃), sulfanyl or fluorosulfanyl (e.g. SFs), C1-C4 alkoxy/ether, ester or carboalkoxy (e.g. OMe, OOMe, or CO₂Me), CN, NO₂, or phenyl or substituted phenyl, with n substituents, preferably selected from C1-C4 alkyl (Me, Et, Pr, Bu), fully or partially fluorinated C1-C4 alkyl (e.g. CF₃), sulfanyl or fluorosulfanyl (e.g. SFs), C1-C4 alkoxy/ether, ester or carboalkoxy (e.g. OMe, OOMe, or CO₂Me), CN, NO₂;
R4 is an alpha carbon of an amino acid, preferably tryptophan, wherein the amine of the amino acid may be acetylated and the carboxyl may be O-methylated, and wherein the amino acid may be a residue of a protein;
R4 is a beta carbon of tryptophan, wherein the tryptophan may be a residue of a protein (with a —CH2- group between the protein backbone and the indole);
R2 is substituted or unsubstituted phenyl;
R3 is H;
R5 is H;
R6 is H;
R7 is H;
R8 is H;
R4 is a residue of a protein;
the indole substrate is a tryptophan substrate, and the method provides a residue-specific bioconjugation strategy for tryptophan-based substrate functionalization;
the indole substrate is a tryptophan substrate of a peptide, a polypeptide, or a protein
the indole substrate is a tryptophan substrate of a peptide, a polypeptide, or a protein and the method results in site- and residue-specific modification of the protein, with applications in synthesis and characterization of antibody-drug conjugates and related biologic therapeutics and imaging agents, chemoproteomics and inhibitor design, as well as modifications to study and improve upon protein function, including solubility, stability, and metabolism and pharmacokinetics;
the indole substrate is a tryptophan substrate of a peptide, a polypeptide, or a protein is an antibody, adeno-associated virus (AAV) capsid protein; the antibody is selected from a single-chain variable fragment antibody, a designed ankyrin repeat proteins (DARPin), and a single variable domain on a heavy chain (VHH) antibody; and/or
the method is combined with stable isotope labeling with amino acid in cell culture (SILAC) or isotope coded affinity tag (ICAT); the method is combined with SILAC or ICAT for quantitative proteomics analysis of tryptophan function in vivo and in vitro by mass spectrometry, with application including but not limited to quantitative analysis of tryptophan reactivity, quantitative analysis of oxidative-sensitive tryptophan, quantitative analysis of C-mannosylated tryptophan and quantitative analysis of C-mannosyltransferase DPY19 substrates.

In an aspect the invention provides a composition comprising an N-sulfonyl oxaziridine of formula I.

In an aspect the invention provides a composition comprising a mixture of an N-sulfonyl oxaziridine of formula I, and an indole substrate of formula II.

In embodiments:
the indole substrate is a tryptophan substrate, preferably a tryptophan residue of a protein.

In an aspect, the invention provides a composition comprising a mixture of an N-sulfonyl oxaziridine of formula I, an indole substrate of formula II, and a cycloadduct of the corresponding formula III.

In an aspect the invention provides a composition of a claim herein in an aqueous, biocompatible medium.

In an aspect the invention provides a strategy for bioconjugation to tryptophan using N-sulfonyl oxaziridine-based reagents that mimic oxidative cyclization reactions on indole natural products to achieve highly selective, rapid, and robust tryptophan labeling. The invention provides broad utility of this method for tryptophan functionalization from proteins to proteomes, including synthesis of antibody-drug conjugates and identification of hyperreactive tryptophan sites in proteomes. Commercial applications include:
1. Therapeutic protein functionalization based on tryptophan bioconjugation such as therapeutic protein pegylation, antibody-drug conjugates, protein labeling for imaging and diagnosis, and alternative protein post translational modifications.
2. Therapeutic peptides functionalization based on tryptophan bioconjugation such as peptide pegylation, peptide-drug conjugates and other peptide post-translational modifications.
3. Therapeutic intervention based on tryptophan bioconjugation for protein function activation and/or inhibition.
4. Biomolecule functionalization based on indole bioconjugation using N-sulfonyl oxaziridine compounds such as DNA, RNA, lipid and sugar bioconjugations.

The invention encompasses all combinations of the particular embodiments recited herein, as if each combination had been laboriously recited.

### DESCRIPTION OF PARTICULAR EMBODIMENTS OF THE INVENTION

Unless contraindicated or noted otherwise, in these descriptions and throughout this specification, the terms “a” and “an” mean one or more, the term “or” means and/or. The examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein, including citations therein, are hereby incorporated by reference in their entirety for all purposes.

The term “alkyl” refers to a hydrocarbon group selected from linear and branched saturated hydrocarbon groups of 1-18, or 1-12, or 1-6 carbon atoms. Examples of the alkyl group include methyl, ethyl, 1-propyl or n-propyl (“n-Pr”), 2-propyl or isopropyl (“i-Pr”), 1-butyl or n-butyl (“n-Bu”), 2-methyl-1-propyl or isobutyl (“i-Bu”), 1-methylpropyl or s-butyl (“s-Bu”), and 1,1-dimethylethyl or t-butyl (“t-Bu”). Other examples of the alkyl group include 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl and 3,3-dimethyl-2-butyl groups.

Lower alkyl means 1-8, preferably 1-6, more preferably 1-4 carbon atoms; lower alkenyl or alkynyl means 2-8, 2-6 or 2-4 carbon atoms.

The term “alkenyl” refers to a hydrocarbon group selected from linear and branched hydrocarbon groups comprising at least one C═C double bond and of 2-18, or 2-12, or 2-6 carbon atoms. Examples of the alkenyl group may be selected from ethenyl or vinyl, prop-1-enyl, prop-2-enyl, 2-methylprop-1-enyl, but-1-enyl, but-2-enyl, but-3-enyl, buta-1,3-dienyl, 2-methylbuta-1,3-diene, hex-1-enyl, hex-2-enyl, hex-3-enyl, hex-4-enyl, and hexa-1,3-dienyl groups.

The term “alkynyl” refers to a hydrocarbon group selected from linear and branched hydrocarbon group, comprising at least one C≡C triple bond and of 2-18, or 2-12, or 2-6 carbon atoms. Examples of the alkynyl group include ethynyl, 1-propynyl, 2-propynyl (propargyl), 1-butynyl, 2-butynyl, and 3-butynyl groups.

The term “cycloalkyl” refers to a hydrocarbon group selected from saturated and partially unsaturated cyclic hydrocarbon groups, comprising monocyclic and polycyclic (e.g., bicyclic and tricyclic) groups. For example, the cycloalkyl group may be of 3-12, or 3-8, or 3-6 carbon atoms. Even further for example, the cycloalkyl group may be a monocyclic group of 3-12, or 3-8, or 3-6 carbon atoms. Examples of the monocyclic cycloalkyl group include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, and cyclododecyl groups. Examples of the bicyclic cycloalkyl groups include those having 7-12 ring atoms arranged as a bicycle ring selected from [4,4], [4,5], [5,5], [5,6] and [6,6] ring systems, or as a bridged bicyclic ring selected from bicyclo[2.2.1]heptane, bicyclo[2.2.2]octane, and bicyclo[3.2.2]nonane. The ring may be saturated or have at least one double bond (i.e. partially unsaturated), but is not fully conjugated, and is not aromatic, as aromatic is defined herein.

The term “aryl” herein refers to a group selected from: 5- and 6-membered carbocyclic aromatic rings, for example, phenyl; bicyclic ring systems such as 7-12 membered bicyclic ring systems wherein at least one ring is carbocyclic and aromatic, selected, for example, from naphthalene, indane, and 1,2,3,4-tetrahydroquinoline; and tricyclic ring systems such as 10-15 membered tricyclic ring systems wherein at least one ring is carbocyclic and aromatic, for example, fluorene.

For example, the aryl group is selected from 5- and 6-membered carbocyclic aromatic rings fused to a 5- to 7-membered cycloalkyl or heterocyclic ring optionally comprising at least one heteroatom selected from N, O, and S, provided that the point of attachment is at the carbocyclic aromatic ring when the carbocyclic aromatic ring is fused with a heterocyclic ring, and the point of attachment can be at the carbocyclic aromatic ring or at the cycloalkyl group when the carbocyclic aromatic ring is fused with a cycloalkyl group. Bivalent radicals formed from substituted benzene derivatives and having the free valences at ring atoms are named as substituted phenylene radicals. Bivalent radicals derived from univalent polycyclic hydrocarbon radicals whose names end in “-yl” by removal of one hydrogen atom from the carbon atom with the free valence are named by adding “-idene” to the name of the corresponding univalent radical, e.g., a naphthyl group with two points of attachment is termed naphthylidene. Aryl, however, does not encompass or overlap with heteroaryl, separately defined below. Hence, if one or more carbocyclic aromatic rings are fused with a heterocyclic aromatic ring, the resulting ring system is heteroaryl, not aryl, as defined herein.

The term “halogen” or “halo” refers to F, Cl, Br or I.

The term “heteroalkyl” refers to alkyl comprising at least one heteroatom.

The term “heteroaryl” refers to a group selected from:
5- to 7-membered aromatic, monocyclic rings comprising 1, 2, 3 or 4 heteroatoms selected from N, O, and S, with the remaining ring atoms being carbon;
8- to 12-membered bicyclic rings comprising 1, 2, 3 or 4 heteroatoms, selected from N, O, and S, with the remaining ring atoms being carbon and wherein at least one ring is aromatic and at least one heteroatom is present in the aromatic ring; and
11- to 14-membered tricyclic rings comprising 1, 2, 3 or 4 heteroatoms, selected from N, O, and S, with the remaining ring atoms being carbon and wherein at least one ring is aromatic and at least one heteroatom is present in an aromatic ring.

For example, the heteroaryl group includes a 5- to 7-membered heterocyclic aromatic ring fused to a 5- to 7-membered cycloalkyl ring. For such fused, bicyclic heteroaryl ring systems wherein only one of the rings comprises at least one heteroatom, the point of attachment may be at the heteroaromatic ring or at the cycloalkyl ring.

When the total number of S and O atoms in the heteroaryl group exceeds 1, those heteroatoms are not adjacent to one another. In some embodiments, the total number of S and O atoms in the heteroaryl group is not more than 2. In some embodiments, the total number of S and O atoms in the aromatic heterocycle is not more than 1.

Examples of the heteroaryl group include, but are not limited to, (as numbered from the linkage position assigned priority 1) pyridyl (such as 2-pyridyl, 3-pyridyl, or 4-pyridyl), cinnolinyl, pyrazinyl, 2,4-pyrimidinyl, 3,5-pyrimidinyl, 2,4-imidazolyl, imidazopyridinyl, isoxazolyl, oxazolyl, thiazolyl, isothiazolyl, thiadiazolyl, tetrazolyl, thienyl, triazinyl, benzothienyl, furyl, benzofuryl, benzoimidazolyl, indolyl, isoindolyl, indolinyl, phthalazinyl, pyrazinyl, pyridazinyl, pyrrolyl, triazolyl, quinolinyl, isoquinolinyl, pyrazolyl, pyrrolopyridinyl (such as 1H-pyrrolo[2,3-b]pyridin-5-yl), pyrazolopyridinyl (such as 1H-pyrazolo[3,4-b]pyridin-5-yl), benzoxazolyl (such as benzo[d]oxazol-6-yl), pteridinyl, purinyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, 1-thia-3,4-diazolyl, furazanyl, benzofurazanyl, benzothiophenyl, benzothiazolyl, benzoxazolyl, quinazolinyl, quinoxalinyl, naphthyridinyl, furopyridinyl, benzothiazolyl (such as benzo[d]thiazol-6-yl), indazolyl (such as 1H-indazol-5-yl) and 5,6,7,8-tetrahydroisoquinoline.

The term “heterocyclic” or “heterocycle” or “heterocyclyl” refers to a ring selected from 4- to 12-membered monocyclic, bicyclic and tricyclic, saturated and partially unsaturated rings comprising at least one carbon atoms in addition to 1, 2, 3 or 4 heteroatoms, selected from oxygen, sulfur, and nitrogen. “Heterocycle” also refers to a 5- to 7-membered heterocyclic ring comprising at least one heteroatom selected from N, O, and S fused with 5-, 6-, and/or 7-membered cycloalkyl, carbocyclic aromatic or heteroaromatic ring, provided that the point of attachment is at the heterocyclic ring when the heterocyclic ring is fused with a carbocyclic aromatic or a heteroaromatic ring, and that the point of attachment can be at the cycloalkyl or heterocyclic ring when the heterocyclic ring is fused with cycloalkyl.

“Heterocycle” also refers to an aliphatic spirocyclic ring comprising at least one heteroatom selected from N, O, and S, provided that the point of attachment is at the heterocyclic ring. The rings may be saturated or have at least one double bond (i.e. partially unsaturated). The heterocycle may be substituted with oxo. The point of the attachment may be carbon or heteroatom in the heterocyclic ring. A heterocyle is not a heteroaryl as defined herein.

Examples of the heterocycle include, but not limited to, (as numbered from the linkage position assigned priority 1) 1-pyrrolidinyl, 2-pyrrolidinyl, 2,4-imidazolidinyl, 2,3-pyrazolidinyl, 1-piperidinyl, 2-piperidinyl, 3-piperidinyl, 4-piperidinyl, 2,5-piperazinyl, pyranyl, 2-morpholinyl, 3-morpholinyl, oxiranyl, aziridinyl, thiiranyl, azetidinyl, oxetanyl, thietanyl, 1,2-dithietanyl, 1,3-dithietanyl, dihydropyridinyl, tetrahydropyridinyl, thiomorpholinyl, thioxanyl, piperazinyl, homopiperazinyl, homopiperidinyl, azepanyl, oxepanyl, thiepanyl, 1,4-oxathianyl, 1,4-dioxepanyl, 1,4-oxathiepanyl, 1,4-oxaazepanyl, 1,4-dithiepanyl, 1,4-thiazepanyl and 1,4-diazepane 1,4-dithianyl, 1,4-azathianyl, oxazepinyl, diazepinyl, thiazepinyl, dihydrothienyl, dihydropyranyl, dihydrofuranyl, tetrahydrofuranyl, tetrahydrothienyl, tetrahydropyranyl, tetrahydrothiopyranyl, 1-pyrrolinyl, 2-pyrrolinyl, 3-pyrrolinyl, indolinyl, 2H-pyranyl, 4H-pyranyl, 1,4-dioxanyl, 1,3-dioxolanyl, pyrazolinyl, pyrazolidinyl, dithianyl, dithiolanyl, pyrazolidinylimidazolinyl, pyrimidinonyl, 1,1-dioxo-thiomorpholinyl, 3-azabicyco[3.1.0]hexanyl, 3-azabicyclo[4.1.0]heptanyl and azabicyclo[2.2.2]hexanyl. Substituted heterocycle also includes ring systems substituted with one or more oxo moieties, such as piperidinyl N-oxide, morpholinyl-N-oxide, 1-oxo-1-thiomorpholinyl and 1, 1-dioxo-1-thiomorpholinyl.

Substituents, such as R1-R8, particularly R1, R3, R5-R8, are selected from: halogen, —R′, —OR′, ═O, ═NR′, ═N—OR′, —NR′R″, —SR′, —SiR′R″R″′, —OC(O)R′, —C(O)R′, —CO₂R′, —CONR′R″, —OC(O)NR′R″, —NR″C(O)R′, —NR′—C(O)NR″R″, —NR′—SO₂NR″′, —NR″CO₂R′, —NH—C(NH₂)═NH, —NR′C(NH₂)═NH, —NH—C(NH₂)═NR′, —S(O)R′, —SO₂R′, —SO₂NR′R″, —NR″SO₂R, —CN and —NO₂, —N₃, —CH(Ph)₂, perfluoro(C1-C4)alkoxy and perfluoro(C1-C4)alkyl, in a number ranging from zero to three, with those groups having zero, one or two substituents being particularly preferred. R, R′, R″ and R′″ each independently refer to hydrogen, unsubstituted (C1-C8)alkyl and heteroalkyl, unsubstituted aryl, aryl substituted with one to three halogens, unsubstituted alkyl, alkoxy or thioalkoxy groups, or aryl-(C1-C4)alkyl groups. When R′ and R″ are attached to the same nitrogen atom, they can be combined with the nitrogen atom to form a 5-, 6- or 7-membered ring. Hence, —NR′R″ includes 1-pyrrolidinyl and 4-morpholinyl, “alkyl” includes groups such as trihaloalkyl (e.g., —CF₃ and —CH₂CF₃), and when the aryl group is 1,2,3,4-tetrahydronaphthalene, it may be substituted with a substituted or unsubstituted (C3-C7)spirocycloalkyl group. The (C3-C7)spirocycloalkyl group may be substituted in the same manner as defined herein for “cycloalkyl”.

Preferred substituents are selected from: halogen, —R′, —OR′, ═O, —NR′R″, —SR′, —SiR′R″R″′, —OC(O)R′, —C(O)R′, —CO₂R′, —CONR′R″, —OC(O)NR′R″, —NR″C(O)R′, —NR″CO₂R′, —NR′—SO₂NR″R″′, —S(O)R′, —SO₂R′, —SO₂NR′R″, —NR″SO₂R, —CN and —NO₂, perfluoro(C1-C4)alkoxy and perfluoro(C1-C4)alkyl, where R′ and R″ are as defined above.

The term “fused ring” herein refers to a polycyclic ring system, e.g., a bicyclic or tricyclic ring system, in which two rings share only two ring atoms and one bond in common. Examples of fused rings may comprise a fused bicyclic cycloalkyl ring such as those having from 7 to 12 ring atoms arranged as a bicyclic ring selected from [4,4], [4,5], [5,5], [5,6] and [6,6] ring systems as mentioned above; a fused bicyclic aryl ring such as 7 to 12 membered bicyclic aryl ring systems as mentioned above, a fused tricyclic aryl ring such as 10 to 15 membered tricyclic aryl ring systems mentioned above; a fused bicyclic heteroaryl ring such as 8- to 12-membered bicyclic heteroaryl rings as mentioned above, a fused tricyclic heteroaryl ring such as 11- to 14-membered tricyclic heteroaryl rings as mentioned above; and a fused bicyclic or tricyclic heterocyclyl ring as mentioned above.

The compounds may contain an asymmetric center and may thus exist as enantiomers. Where the compounds possess two or more asymmetric centers, they may additionally exist as diastereomers. Enantiomers and diastereomers fall within the broader class of stereoisomers. All such possible stereoisomers as substantially pure resolved enantiomers, racemic mixtures thereof, as well as mixtures of diastereomers are intended to be included. All stereoisomers of the compounds and/or pharmaceutically acceptable salts thereof are intended to be included. Unless specifically mentioned otherwise, reference to one isomer applies to any of the possible isomers. Whenever the isomeric composition is unspecified, all possible isomers are included.

The compounds of the invention may also contain unnatural proportions of atomic isotopes at one or more of the atoms that constitute such compounds, such as deuterium, e.g. —CD₃, CD₂H or CDH₂ in place of methyl. For example, the compounds may be radiolabeled with radioactive isotopes, such as for example tritium (³H), iodine-125 (¹²⁵I) or carbon-14 (¹⁴C). All isotopic variations of the compounds of the invention, whether radioactive or not, are intended to be encompassed within the scope of the invention.

### Redox-Based Method for Tryptophan Functionalization Through Bioinspired Oxidative Cyclization.

Inspired by oxidative cyclization reactions on L-tryptophan that generate alkaloid natural products with a versatile hexahydropyrrolo[2,3-b]indole scaffold (27), we reasoned that such a redox-based approach might open a new avenue for tryptophan modification over conventional acid-base chemistry. As a starting point for developing oxidative cyclization warheads for tryptophan bioconjugation, we observed that N-alkoxycarbonyl oxaziridines, which exerted rapid reactivity and high biocompatibility for oxidative methionine functionalization from proteins to proteomes, had negligible reactivity for tryptophan oxidation (4). Indeed, molecular orbital calculations revealed that tryptophan is more difficult to oxidize relative to methionine, as the highest occupied molecular orbital (HOMO) energy of tryptophan (−7.45 eV) is higher than that of methionine (−8.25 eV). These results pointed to the need to develop more reactive oxaziridine reagents for redox-based modification of tryptophan. We then calculated lowest unoccupied molecular orbital (LUMO) energies of several diverse classes of oxaziridines and found that the N-sulfonyl oxaziridine motif can accept electrons the most readily.

Initial reactivity studies with the N-sulfonyl oxaziridine Ox-W1 and N-acetyl-L-tryptophan methyl ester (Ac-Trp-OMe) were then performed in PBS/organic solvent mixture for 10 min at room temperature and subjected to liquid chromatography-mass spectrometry (LC-MS) analysis. Tryptophan reacted with a ˜50% conversion. Two major products were observed: the desired indole cycloadduct and hydroxyl tryptophan. The cycloadduct was generated with 28% yield, consistent with simple indoles that can react with such oxaziridines in organic solution (28). Moreover, the reaction of Ox-W1 with methionine could only generate the traceless methionine sulfoxide adduct. We then synthesized and screened a library of 20 different oxaziridines for Trp-CLiC chemistry; Ox-W10, Ox-W17, and Ox-W19 emerged as the top lead candidates with improved ˜90% selectivity and high reactivity for the indole cycloadduct product, with a measured reaction rate of 24.9±1.3M⁻¹ s⁻¹ that is on par with click chemistry reactions (29-31).

### Oxidative Tryptophan Bioconjugation on Peptides, Proteins, and Proteomes.

With lead oxaziridine candidates (Ox-W16 to Ox-W21) in hand that bear synthetic handles for subsequent addition of functional payloads via copper-mediated alkyne-azide cyclization (CuAAC) chemistry, we evaluated these Trp-CLiC reagents for tryptophan modification of peptides, proteins, and proteomes. GLP-1, which has a single tryptophan site, was chosen as an initial model peptide substrate. From reaction screens between GLP-1 (50 μM) and oxaziridine (300 μM), Ox-W18 which has best solubility showed the highest cycloadduct yield (74%) with quantitative conversion. Similar high selectivity and efficiency for tryptophan labeling was observed on Glucagon as a second model peptide substrate, which has one tryptophan and one methionine site. We also observed formation of methionine sulfoxide on Glucagon from oxygen-atom transfer oxidation as a traceless side product. We then went on to label the model protein IL8, which has one partially buried tryptophan site. To our delight, this tryptophan residue could be modified only after treatment with a denaturant to increase its solvent accessibility, and liquid chromatography with tandem mass spectrometry (LC-MS/MS) analysis confirmed formation of the oxidative cycloadduct product. Finally, we tested the reactivity and selectivity of tryptophan labeling with Ox-W18 in whole proteomes using human embryonic kidney 293T (HEK 293T) cell lysates. Trypsin digestion with subsequent LC-MS/MS analysis established that Ox-W18 has high selectivity for tryptophan modification in proteomes (32). Taken together, the data indicate that Trp-CLiC is an effective strategy for tryptophan bioconjugation across peptide, protein, and proteome levels, with N-sulfonyl oxaziridine derivatives emerging as privileged reagent scaffolds for promoting oxidative cyclization reactions on tryptophan to yield the corresponding indole cycloadduct products.

### Oxidative Tryptophan Bioconjugation is Rapid and Targets Solvent-Accessible Surface Tryptophan Sites.

After successfully demonstrating tryptophan labeling on peptides, proteins, and proteomes using Trp-CLiC, we sought to characterize features of this novel bioconjugation method in more depth using lysozyme as a model protein substrate that contains six distinctive tryptophan residues. Because of its indole ring, tryptophan exhibits a unique absorption signature centered around 280 nm, which is commonly applied to measure protein concentrations (33). Based on spectroscopic analyses, we used the ratio of absorption signatures at 260 and 280 nm (A260/A280 ratio) to track tryptophan bioconjugation on lysozyme. Time-dependent modification of lysozyme with Ox-W18 (1 mg/mL denatured protein, 1 mM Ox reagent) monitored by A260/A280 ratio changes showed completion of the reaction in less than two minutes. In-gel fluorescence imaging assays on modified lysozyme and bovine serum albumin (BSA) after Ox-W18 labeling, followed by subsequent click functionalization with fluorescent dyes, further confirmed the rapid reactivity of Ox-W18 towards tryptophan.

We next evaluated how many and which tryptophan residues on lysozyme are prioritized for redox-based Trp-CLiC labeling under native folded conditions. The observed A260/A280 ratio was 0.80, which correlated to a single tryptophan residue being labeled. Moreover, in-gel fluorescence imaging after denaturing showed that both the protein fluorescence signal and molecular weight increase, consistent with a greater number of tryptophan residues modified upon protein unfolding. We then moved on to identify which tryptophan sites are targeted and tested the hypothesis that the propensity of oxidative tryptophan modification reactivity might correlate with solvent accessibility. To this end, we used the Discovery Studio platform to calculate the relative solvent accessibility of the six tryptophan residues on lysozyme. Trypsin digestion and LC-MS/MS analysis of Ox-W18-labeled lysozyme revealed that W62, the most surface-exposed tryptophan site, was also the most targeted site for native labeling from the labeled/unlabeled peptide count ratio. In contrast, the adjacent W63 site could only be modified with the Ox-W18 reagent after denaturing the protein to leverage its solvent accessibility. These data establish that the Trp-CLiC oxidative cyclization method shows preferential labeling of surface-exposed surface tryptophan residues.

### Oxidative Tryptophan Bioconjugation Enables Modification of Engineered Antibody Scaffolds and Detection of Stress-Induced Protein Unfolding.

As the Trp-CLiC method enables rapid and specific tryptophan bioconjugation and preferentially targets surface-exposed tryptophan residues, we sought to exploit this selectivity for various protein labeling applications. As a first example to showcase the Trp-CLiC approach in this context, we introduced tryptophan residues into solvent-accessible locations for single-site-selective bioconjugation of antibody scaffolds. First, the solvent accessibilities of native tryptophan residues within Her2-Fab were analyzed, indicating that all endogenous tryptophan residues in this scaffold appear to be partially or totally buried. With this information in hand, we engineered two Her2-Fab constructs (Her2-Fab T74W and T198W), each possessing one de novo tryptophan residue distributed over the surface of the antibody. Indeed, the Trp-CLiC reagent showed a clear preference to target solvent-accessible engineered tryptophan residue with high selectivity and reactivity (>80%). In contrast, negligible bioconjugation was detected on wildtype Her2-Fab. The modest stability of such constructs precluded further evaluation in vivo, but the results provide a starting point for antibody functionalization via site-specific tryptophan modification.

In another application, we used Trp-CLiC as a general method to probe stress-induced protein unfolding. Indeed, under various environmental stresses, folded proteins will expose more hydrophobic residues as they unfold, and accumulation and aggregation of unfolded proteins is regarded as a signal of loss of proteostasis (34-37). Owing to its sterically demanding and hydrophobic properties, tryptophan residues are more likely to be buried in protein cores. We reasoned that this oxidative cyclization method could enable monitoring of increases in solvent accessibility of buried tryptophan residues during stress-induced protein unfolding. To test this hypothesis, we labeled lysozyme as a model protein in the presence of varying concentrations of guanidinium chloride (GdmCl) as a denaturant. As anticipated, changes in A260/A280 ratios consistent with tryptophan labeling were observed in a dose-dependent manner with increasing GdmCl concentrations without protein sequence bias, deciphering quantitative unfolding transitions which fit the three-state model. We then used Trp-CLiC to monitor stress-induced protein unfolding in whole cell proteomes. HeLa cells were treated with the proteasome inhibitor MG-132, the ER stress inducer Tunicamycin to trigger unfolded protein stress responses, or vehicle control, then lysed and labeled with N-sulfonyl oxaziridine linked to desthiobiotin. Indeed, more desthiobiotin staining signal was observed in environmentally stressed cells compared to control counterparts, consistent with higher levels of tryptophan labeling due to increased solvent exposure of internal tryptophan residues during protein unfolding.

### Oxidative Tryptophan Bioconjugation Enables Activity-Based Profiling of Functional Tryptophan Sites in Proteomes.

We next moved on to apply Trp-CLiC at the whole proteome level, specifically to identify new functional tryptophan sites in cells using activity-based protein profiling (ABPP). HEK 293T cell lysates were treated with the Ox-W18 probe in a standard ABPP workflow, followed by installation of acid-cleavable biotin enrichment tag using CuAAC click chemistry, capture with streptavidin-labeled magnetic beads, on-bead trypsin digestion, and LC-MS/MS analysis of probe-modified peptides. Interestingly, we observed that the oxidative indole cycloadduct product after acid-cleavage is collision-induced dissociation (CID)-cleavable, providing two reporter ions at m/z: 213.17 and 425.16 that could be used to selectively identify the targeted tryptophan sites with higher confidence.

From the ABPP studies with the Trp-CLiC method, 190 unique hyperreactive tryptophan sites from 151 probe-modified proteins were identified in more than two replicates. These Ox-W18-labeled tryptophan sites were found in many classes of proteins, including enzymes, chaperones, and nucleoproteins, as well as many structural proteins. Meanwhile, we noticed that many targets are classified as non-druggable and highly enriched in diverse phase-separated compartments, such as the nucleolus, nuclear body, paraspeckle and stress granule. Moreover, global analysis of hyperreactive tryptophan sites identified by Trp-CLiC ABPP showed a preference for surface-exposed tryptophan residues. For example, the hyperreactive W196 site identified on GAPDH, W346 site found on TBA1B, and W324 and W148 on LDHA enzyme are the most surface-exposed tryptophan residues in these proteins, whereas other tryptophan residues were not probed. Interestingly, the W196 site on GADPH is a crucial residue, as the corresponding W196F mutant leads to increased free radical-induced aggregation of this protein target (34). GAPDH and TBA1B were chosen as representative examples to showcase the Trp-CLiC method for ABPP. Increased Ox-W18 addition resulted in more GAPDH and TBA1B labeling as determined by Western Blot analysis. We then sought to further investigate the character of probed solvent-accessible tryptophan sites by performing motif analysis. Interestingly, we observed that lysine is highly enriched in sites near probe-modified tryptophan residues. Lysine is hydrophilic and has the capacity to form cation-π interactions with the indole tryptophan ring, providing a potential pathway for stabilizing hydrophobic tryptophan residues being surface-exposed (38, 39).

As we also observed that the probe-modified proteins from Trp-CLiC ABPP are highly enriched in phase-separated subcellular compartments, we hypothesized that this method could be employed to identify not only proteins involved in phase separation but specific functional tryptophan sites on those protein targets, where privileged, surface-exposed tryptophan residues could mediate protein phase separation and mutations of such sites could trigger disease-relevant dysregulation of physiological compartmentalization. In this context, NPM1 is the scaffold protein of granular component subcompartment of the nucleolus, and mutations of the two highly conserved tryptophan residues (W288 and W290) located on the C-terminal RRM domain surface are the most common genetic lesions found in acute myeloid leukemia (40, 41). Indeed, Western blot analysis detected decreased or negligible fluorescence signal after either or both tryptophan residues were mutated to alanine, indicating that these two tryptophan sites are essential for NPM1 labeling by the Trp-CLiC method. We then proceeded to perform fluorescence imaging assays in cells to monitor functional changes in NPM1 distributions between wildtype and alanine mutants. Interestingly, mutation of these C-terminal tryptophan sites resulted in an alteration of NPM1 localization and phase separation, leading to a more diffuse fluorescence signal detected across nucleus matrix that identified the importance of these functional residues. As a second example to showcase this Trp-CLiC method for identification of functional tryptophan sites in mediating phase separation, NONO protein is the necessary factor for paraspeckle formation and the mutation of the conserved W271 is related to mental retardation. (42) W271 was also Trp-CLiC targeted and mutation of this single residue also markedly influenced NONO-mediated phase separation and paraspeckle formation. In detail, we observed a significant decrease in punctate-positive cells in W271A mutant relative to wildtype, but in W271A cells that did form phase separated compartments, their puncta size was abnormally large compared to the wildtype congeners. As such, the Trp-CLiC method provides an effective strategy for discovering and characterizing functional tryptophan residues in proteomes and their potential roles in disease-relevant processes.

Trp-CLiC provides a unique and general redox-based method that enables chemoselective tryptophan bioconjugation to complement traditional acid-base strategies for more nucleophilic amino acids such as cysteine and lysine. Biosynthesis of indole-based alkaloids provides inspiration for the development of oxaziridine reagents that enable selective and rapid oxidative cyclization on indole side chains of tryptophan from proteins to proteomes. Despite its rarity in eukaryotic organisms, tryptophan plays crucial roles in stabilizing protein secondary structures, promoting enzymatic activity, and mediating protein-protein interactions. Moreover, post-translational modifications such as tryptophan oxidation and C-mannosylation also contribute to signal transduction and protein secretion. Indeed, in this report we highlighted the use of Trp-CLiC to identify and characterize new functional disease-related tryptophan sites that regulate protein-mediated phase separation processes. Owing to its rapid and specific tryptophan redox reactivity with a preference for solvent-accessible surface tryptophan sites, Trp-CLiC offers a broadly useful chemical platform for precise addition of payloads to proteins, identifying and characterizing functional tryptophans in whole proteomes, and a starting point for therapeutic strategies that engage tryptophan reactivity via activation and/or inhibition.

### REFERENCES

1. S. Barik, The Uniqueness of Tryptophan in Biology: Properties, Metabolism, Interactions and Localization in Proteins. Int. J. Mol. Sci. 21, 8776-8797, (2020).
2. J. J. Hu et al, Chemical modifications of tryptophan residues in peptides and proteins. J. Pept. Sci. 27, e3286-3295, (2021).
3. C. D. Spicer, B. G. Davis, Selective chemical protein modification. Nat. Commun. 5, 4740-4753, (2014).
4. J. N. deGruyter et al, Residue-Specific Peptide Modification: A Chemist's Guide. Biochemistry 56, 3863-3873, (2017).
5. S. Lin et al, Redox-based reagents for chemoselective methionine bioconjugation. Science 355, 597-602, (2017).
6. E. A. Hoyt et al, Contemporary approaches to site-selective protein modification. Nat. Rev. Chem. 3, 147-171, (2019).
7. A. A. Bogan, K. S. Thorn, Anatomy of hot spots in protein interfaces. J. Mol. Biol. 280, 1-9, (1998).
8. G. L. Dignon, R. B. Best, J. Mittal, Biomolecular Phase Separation: From Molecular Driving Forces to Macroscopic Properties. Annu. Rev. Phys. Chem. 71, 53-75, (2020).
9. S. N. Murthy et al, Conserved tryptophan in the core domain of transglutaminase is essential for catalytic activity. Proc. Natl. Acad. Sci. U.S.A. 99, 2738-2742, (2002).
10. Y. Guo et al, Protein structure. Structure and activity of tryptophan-rich TSPO proteins. Science 347, 551-555, (2005).
11. H. B. Gray, J. R. Winkler, Hole hopping through tyrosine/tryptophan chains protects proteins from oxidative damage. Proc. Natl. Acad. Sci. U.S.A. 112, 10920-10925, (2015).
12. M. Orita et al, Coumarin and chromen-4-one analogues as tautomerase inhibitors of macrophage migration inhibitory factor: discovery and X-ray crystallography. J. Med. Chem. 44, 540-547, (2001).
13. B. Campanini et al, Surface-exposed tryptophan residues are essential for O-acetylserine sulfhydrylase structure, function, and stability. J. Biol. Chem. 278, 37511-37519, (2003).
14. S. W. Taylor et al, Oxidative post-translational modification of tryptophan residues in cardiac mitochondrial proteins. J. Biol. Chem. 278, 19587-19590, (2003).
15. R. Helland et al, An oxidized tryptophan facilitates copper binding in Methylococcus capsulatus-secreted protein MopE. J. Biol. Chem. 283, 13897-13904, (2008).
16. M. Ehrenshaft et al, Tripping up Trp: Modification of protein tryptophan residues by reactive oxygen species, modes of detection, and biological consequences. Free Radical Biol. Med. 89, 220-228, (2015).
17. A. John et al, Yeast- and antibody-based tools for studying tryptophan C-mannosylation. Nat. Chem. Biol. 17, 428-437, (2021).
18. A. Shcherbakova et al, C-mannosylation supports folding and enhances stability of thrombospondin repeats. Elife 8, e52978-52972, (2019).
19. J. M. Antos et al, Chemoselective tryptophan labeling with rhodium carbenoids at mild pH. J. Am. Chem. Soc. 131, 6301-6308, (2009).
20. B. V. Popp, Z. T. Ball, Structure-Selective Modification of Aromatic Side Chains with Dirhodium Metallopeptide Catalysts. J. Am. Chem. Soc. 132, 6660-6662, (2010).
21. J. Ruiz-Rodriguez, F. Albericio, R. Lavilla, Postsynthetic modification of peptides: chemoselective C-arylation of tryptophan residues. Chem.-Eur. J. 16, 1124-1127, (2010).
22. M. B. Hansen et al, Chemo- and Regioselective Ethynylation of Tryptophan-Containing Peptides and Proteins. Chem.-Eur. J. 22, 1572-1576, (2016).
23. Y. Seki et al, Transition Metal-Free Tryptophan-Selective Bioconjugation of Proteins. J. Am. Chem. Soc. 138, 10798-10801, (2016).
24. Y. Yu et al, Chemoselective Peptide Modification via Photocatalytic Tryptophan beta-Position Conjugation. J. Am. Chem. Soc. 140, 6797-6800, (2018).
25. S. J. Tower et al, Selective Modification of Tryptophan Residues in Peptides and Proteins Using a Biomimetic Electron Transfer Process. J. Am. Chem. Soc. 142, 9112-9118, (2020).
26. M. Imiolek et al, Residue-Selective Protein C-Formylation via Sequential Difluoroalkylation-Hydrolysis. ACS Cent. Sci. 7, 145-155, (2021).
27. A. Roy et al, Hexahydropyrrolo-[2,3-b]-indole alkaloids of biological relevance: proposed biosynthesis and synthetic approaches. Arkivoc 1, 437-471, (2020).
28. S. Mithani et al, An anomalous reaction of 2-benzenesulfonyl-3-aryloxaziridines (Davis reagents) with indoles: Evidence for a stepwise reaction of the Davis reagent with a π-bond. J. Am. Chem. Soc., 11, 1159-1160, (1997).
29. V. V. Rostovtsev et al, A stepwise huisgen cycloaddition process: copper (I)-catalyzed regioselective “ligation” of azides and terminal alkynes. Angew. Chem. Int. Ed. 114, 2708-2711, (2002).
30. N. J. Agard, J. et al, A strain-promoted [3+2] azide-alkyne cycloaddition for covalent modification of biomolecules in living systems. J. Am. Chem. Soc. 126, 15046-15047, (2004).
31. K. Lang, J. W. Chin, Bioorthogonal reactions for labeling proteins. ACS Chem. Biol. 9, 16-20, (2014).
32. P. R. A. Zanon et al, Profiling the Proteome-Wide Selectivity of Diverse Electrophiles. ChemRxiv https://chemrxiv.org/engage/chemrxiv/article-details/60c755f2bb8c1a7d393dc505 (2021).
33. H. Edelhoch, Spectroscopic determination of tryptophan and tyrosine in proteins. Biochemistry 6, 1948-1954, (1967).
34. C. Hetz et al, Targeting the unfolded protein response in disease. Nat. Rev. Drug Discovery 12, 703-719, (2013).
35. M. Z. Chen et al, A thiol probe for measuring unfolded protein load and proteostasis in cells. Nat. Commun. 8, 474-483, (2017).
36. E. J. Walker et al, Global analysis of methionine oxidation provides a census of folding stabilities for the human proteome. Proc. Natl. Acad. Sci. U.S.A. 116, 6081-6090, (2019).
37. A. L. Samson et al, Oxidation of an Exposed Methionine Instigates the Aggregation of Glyceraldehyde-3-phosphate Dehydrogenase. J. Biol. Chem. 289, 26922-26936, (2014).
38. J. C. Ma, D. A. Dougherty, The cation-π interaction. Chem. Rev. 97, 1303-1324, (1997).
39. J. P. Gallivan, D. A. Dougherty, Cation-π interactions in structural biology. Proc. Natl. Acad. Sci. U.S.A. 96, 9459-9464, (1999).
40. C. G. Grummitt et al, Structural Consequences of Nucleophosmin Mutations in Acute Myeloid Leukemia. J. Biol. Chem. 283, 23326-23332, (2008).
41. D. L. J. Lafontaine et al, The nucleolus as a multiphase liquid condensate. Nat. Rev. Mol. Cell Biol. 22, 165-182, (2020).
42. D. M. Passon et al, Structure of the heterodimer of human NONO and paraspeckle protein component 1 and analysis of its role in subnuclear body formation. Proc. Natl. Acad. Sci. U.S.A. 109, 4846-4850, (2012).

### 1. Synthesis of N-Sulfonyl Oxaziridine Library

### 1.1 Characterization of Indole Cycloadduct

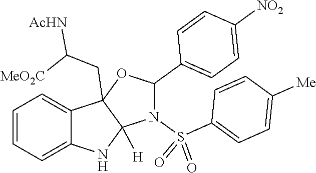 [SMILES: COC(=O)C(CC12OC(c3ccc([N+](=O)[O-])cc3)N(S(=O)(=O)c3ccc(C)cc3)C1Nc1ccccc12)NC(C)=O]

To a stirring suspension of oxaziridine (160 mg, 0.5 mmol, 1.0 equiv.) in MeCN (3 mL) was added Ac-Trp-OMe (130 mg, 0.5 mmol, 1.0 equiv.) as a solution in MeOH (2 mL). Additional MeOH (1 mL) was used to quantitatively transfer the reagent. The mixture was stirred at room temperature for 16 h, then the solvent removed in vacuo. Purification by silica gel chromatography afforded the desired cycloadduct (37 mg, 13%) as a white solid. One diastereomer could be isolated (18 mg, 10:1 d.r.) and characterized below.

¹H NMR (600 MHz, MeOD) δ 7.95-7.89 (m, 2H), 7.48-7.42 (m, 2H), 7.27-7.23 (m, 2H), 7.21 (m, 1H), 7.17 (m, 1H), 7.07 (d, J=8.0 Hz, 2H), 6.77 (t, J=7.5 Hz, 1H), 6.68 (d, J=8.0 Hz, 1H), 5.92 (s, 1H), 5.48 (s, 1H), 4.95 (dd, J=9.5, 3.1 Hz, 1H), 3.71 (s, 3H), 2.76 (dd, J=15.1, 3.1 Hz, 1H), 2.36-2.27 (m, 4H), 1.97 (s, 3H).

¹³C NMR (151 MHz, MeOD) δ 173.6, 173.3, 151.4, 150.1, 145.3, 142.4, 138.9, 132.0, 131.7, 130.2, 128.6, 127.6, 125.2, 123.8, 120.2, 110.7, 92.1, 89.5, 80.8, 53.1, 38.4, 22.6, 21.4.

HRMS (ESI): Calculated for C₂₈H₂₉N₄O₈S [M+H]+: 581.1701. Found 581.1727.

### 1.2 Synthesis of N-Sulfonyl Oxaziridine Library

### General Procedure A

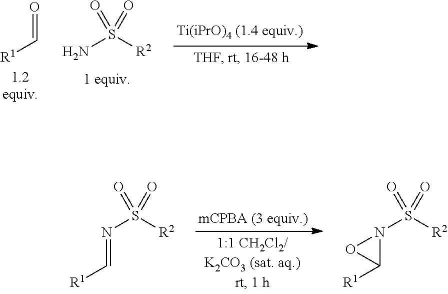

To a solution of sulfonamide (10 mmol, 1.0 equiv.) and aldehyde (12 mmol, 1.2 equiv.) in dry THF (30 mL) was added Ti(iPrO)₄ (4.1 mL, 14 mmol, 1.4 equiv.) dropwise at room temperature. The reaction was monitored by 1H NMR using small aliquots dissolved in C₆D₆. After 16-48 h, the reaction mixture was diluted with CH₂Cl₂ (50 mL) and water (50 mL), and extracted with additional CH₂Cl₂ (3×50 mL). The combined organic phases were concentrated in vacuo until a total volume of ˜40 mL to afford a solution of crude imine which was used directly in the next step.

In a separate round-bottom flask, mCPBA (75%, 6.9 g, 30 mmol, 3.0 equiv.) was prestirred in 1:1 CH₂Cl₂/sat. aq. K₂CO₃ (60 mL) at room temperature for 10 minutes. The solution of crude imine from the previous step was added via addition funnel over 5 minutes, using additional CH₂Cl₂ (10 mL) rinses for a quantitative transfer. The reaction was monitored by TLC with staining with phosphomolybdic acid (PMA); the oxaziridine product stains as a distinctively dark spot. After completion of the reaction (˜1 h), the reaction was diluted with CH₂Cl₂ (50 mL) and washed with sat. aq. NaHCO₃ (30 mL) and brine (30 mL). The combined organic layers were dried over Na₂SO₄, filtered and concentrated in vacuo. Purification by column chromatography afforded the corresponding oxaziridine.

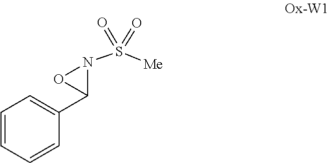 [SMILES: CS(=O)(=O)N1OC1c1ccccc1]

General Procedure A was followed starting from methanesulfonamide (951 mg, 10 mmol, 1.0 equiv.) and benzaldehyde (1.2 mL, 12 mmol, 1.2 equiv.). Purification by column chromatography (0% to 30% EtOAC/hexane) afforded the corresponding oxaziridine (437 mg, 22%) as a white solid. Spectral data matched literature reports.¹

¹H NMR (300 MHz, CDCl₃) δ 7.55-7.38 (m, 5H), 5.46 (s, 1H), 3.23 (s, 3H).

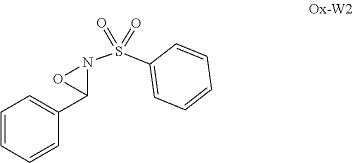 [SMILES: O=S(=O)(c1ccccc1)N1OC1c1ccccc1]

(E)-N-benzylidenebenzenesulfonamide (1.0 g, 4.1 mmol, 1.0 equiv.) and NEt₃BnCl (112 mg, 0.49 mmol, 0.12 equiv.) were taken up in CHCl₃ (4 mL) and sat. aq. NaHCO₃ (4 mL). mCPBA (75%, 1.125 g, 4.89 mmol, 1.2 equiv.) added dropwise as a solution in CH₂Cl₂ (10 mL) over 30 minutes and the reaction was stirred at room temperature for 3 h. The organic layer was separated and washed with 10% Na₂SO₃, sat. aq. NaHCO₃, brine, dried over Na₂SO₄, filtered and concentrated in vacuo. Purification by column chromatography (0% to 20% EtOAc/Hex) afforded the corresponding oxaziridine (749 mg, 70%) as a white solid. Spectral data matched literature reports.²

¹H NMR (300 MHz, CDCl₃) δ 8.10-8.03 (m, 2H), 7.76 (d, J=7.4 Hz, 1H), 7.64 (dd, J=8.5, 7.1 Hz, 2H), 7.53-7.37 (m, 5H), 5.49 (s, 1H).

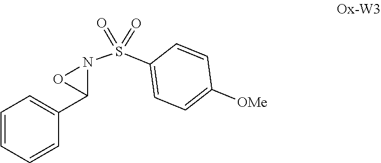 [SMILES: COc1ccc(S(=O)(=O)N2OC2c2ccccc2)cc1]

General Procedure A was followed starting from 4-methoxyphenylsulfonamide (1.1 g, 5.9 mmol, 1.0 equiv.) and benzaldehyde (0.73 mL, 7.1 mmol, 1.2 equiv.). Purification by column chromatography (0% to 40% EtOAc/hexane) afforded the corresponding oxaziridine (1.1 g, 37%) as a white solid. Spectral data matched literature reports.³

¹H NMR (300 MHz, CDCl₃) δ 8.03-7.93 (m, 2H), 7.53-7.34 (m, 5H), 7.13-7.04 (m, 2H), 5.43 (s, 1H), 3.92 (s, 3H).

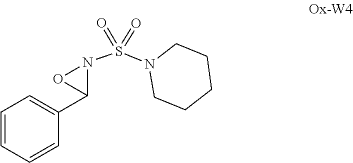 [SMILES: O=S(=O)(N1CCCCC1)N1OC1c1ccccc1]

Ox-W4 was prepared according to the standard procedure. The crude material was initially purified on a silica gel Biotage column (25G) with a gradient of 2-20% EtOAc/hexanes, but remained partially impure by TLC analysis. A portion of this material was dissolved in CH₂Cl₂, loaded onto and purified on a preparatory plate (silica gel base) using 10% EtOAc/hexanes as the mobile phase. A clear color oil which crystallized to form a white solid (294 mg, 1.1 mmol, 37% yield from 3 mmol of sulfonamide starting material) was obtained upon storage at −20° C.

¹H NMR (400 MHz, Chloroform-d) δ 7.63-7.37 (m, 5H), 5.28 (s, 1H), 3.58-3.43 (m, 4H), 1.79-1.68 (m, 4H), 1.68-1.59 (m, 2H). (Contains ˜5% of the aldehyde starting material as an impurity).

¹³C NMR (126 MHz, CDCl₃) δ 131.3, 131.3, 128.9, 128.3, 76.4, 48.0, 25.5, 23.7.

HRMS: Calculated for C₁₂H₁₆N₂O₃SNa [M+Na]⁺=291.0774. Measured [M+Na]⁺=291.0749

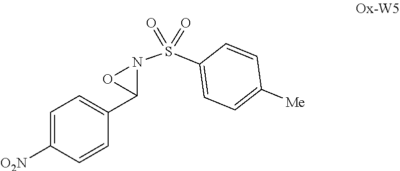 [SMILES: Cc1ccc(S(=O)(=O)N2OC2c2ccc([N+](=O)[O-])cc2)cc1]

General Procedure A was followed starting from 4-methylbenzenesulfonamide (1.71 g, 10 mmol, 1.0 equiv.) and 4-nitrobenzaldehyde (1.81 g, 12 mmol, 1.2 equiv.). Purification by column chromatography (0% to 40% EtOAc/hexane) afforded the corresponding oxaziridine (324 mg, 11%) as a white solid. Spectral data matched literature reports.⁴

¹H NMR (300 MHz, CDCl₃) δ 8.31-8.22 (m, 2H), 7.93 (d, J=8.3 Hz, 2H), 7.64 (d, J=8.8 Hz, 2H), 7.45 (d, J=8.1 Hz, 2H), 5.56 (s, 1H), 2.51 (s, 3H).

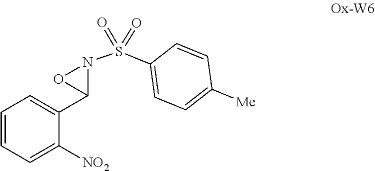 [SMILES: Cc1ccc(S(=O)(=O)N2OC2c2ccccc2[N+](=O)[O-])cc1]

General Procedure A was followed starting from 4-methylbenzenesulfonamide (1.71 g, 10 mmol, 1.0 equiv.) and 2-nitrobenzaldehyde (1.81 g, 12 mmol, 1.2 equiv.). Purification by column chromatography (20% to 100% EtOAc/hexane) afforded the corresponding oxaziridine (1.8 g, 55%) as a white solid. Spectral data matched literature reports.⁵

¹H NMR (300 MHz, CDCl₃) δ 8.23 (dd, J=8.1, 1.4 Hz, 1H), 8.02-7.92 (m, 2H), 7.75-7.55 (m, 3H), 7.43 (m, 2H), 5.98 (s, 1H), 2.48 (s, 3H).

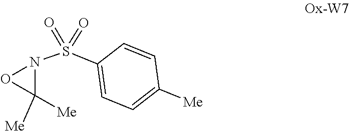 [SMILES: Cc1ccc(S(=O)(=O)N2OC2(C)C)cc1]

A modified General Procedure A was followed starting from 4-methylbenzenesulfonamide (2.4 g, 14 mmol, 1.0 equiv.), dry acetone (6 mL), and Ti(iPrO)₄ (10 mL, 34 mmol, 2.4 equiv.) in CH₂Cl₂ (20 mL). Purification by column chromatography (10% EtOAc/hexane) afforded the corresponding oxaziridine (256 mg, 8%) as a colorless oil. Spectral data matched literature reports.⁶

¹H NMR (300 MHz, CDCl₃) δ 7.93-7.83 (m, 2H), 7.43-7.32 (m, 2H), 2.46 (s, 3H), 2.08 (s, 3H), 1.56 (s, 3H).

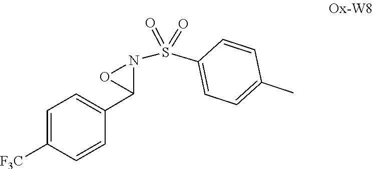 [SMILES: Cc1ccc(S(=O)(=O)N2OC2c2ccc(C(F)(F)F)cc2)cc1]

General Procedure A was followed starting from 4-methylbenzenesulfonamide (1.71 g, 10 mmol, 1.0 equiv.) and 4-trifluoromethylbenzaldehyde (1.6 mL, 12 mmol, 1.2 equiv.). Purification by column chromatography (0% to 20% EtOAc/hexane) afforded the corresponding oxaziridine (1.9 g, 56%) as a white solid. Spectral data matched literature reports.⁷

¹H NMR (300 MHz, CDCl₃) δ 7.98-7.88 (m, 2H), 7.67 (d, J=8.2 Hz, 2H), 7.62-7.52 (m, 2H), 7.50-7.39 (m, 2H), 5.51 (s, 1H), 2.51 (s, 3H).

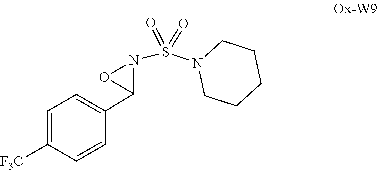 [SMILES: O=S(=O)(N1CCCCC1)N1OC1c1ccc(C(F)(F)F)cc1]

General Procedure A was followed starting from piperidine-1-sulfonamide (1.6 g, 10 mmol, 1.0 equiv.) and 4-trifluoromethylbenzaldehyde (1.6 mL, 12 mmol, 1.2 equiv.). Purification by column chromatography (0% to 20% EtOAc/hexane) afforded the corresponding oxaziridine (2.6 g, 77%) as a white solid.

¹H NMR (300 MHz, CDCl₃) δ 7.68 (d, J=8.3 Hz, 2H), 7.59 (d, J=8.2 Hz, 2H), 5.33 (s, 1H), 3.51 (dd, J=6.3, 4.5 Hz, 4H), 1.79-1.55 (m, 6H).

¹³C NMR (126 MHz, CDCl₃) δ 135.2, 133.3 (q, J=32.5 Hz), 128.7, 125.9 (q, J=3.8 Hz), 123.7 (q, J=273 Hz), 75.3, 48.04, 25.5, 23.6.

¹⁹F NMR (376 MHz, CDCl₃) δ −62.2.

HRMS (ESI): Calculated for C₁₃H₁₉F₃N₃O₃S [M+NH₄]+: 354.1093. Found 354.1071.

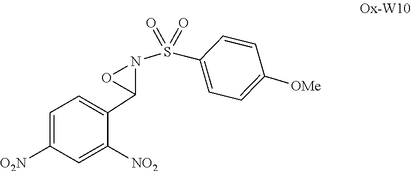 [SMILES: COc1ccc(S(=O)(=O)N2OC2c2ccc([N+](=O)[O-])cc2[N+](=O)[O-])cc1]

General Procedure A was followed starting from 4-methoxybenzenesulfonamide (562 mg, 3 mmol, 1.0 equiv.) and 2,4-dinitrobenzaldehyde (706 mg, 3.6 mmol, 1.2 equiv.). Purification by column chromatography (10% to 100% EtOAc/hexane), followed by trituration with 1:1 pentane/Et₂O afforded the corresponding oxaziridine (281 mg, 25%) as a white solid.

¹H NMR (300 MHz, CDCl₃) δ 9.06 (d, J=2.3 Hz, 1H), 8.54 (dd, J=8.6, 2.3 Hz, 1H), 8.07-7.96 (m, 2H), 7.85 (d, J=8.6 Hz, 1H), 7.15-7.04 (m, 2H), 6.06 (s, 1H), 3.93 (s, 3H).

¹³C NMR (126 MHz, CDCl₃) δ 165.5, 149.2, 148.4, 134.0, 132.6, 131.0, 128.7, 123.9, 120.6, 115.0, 74.8, 56.1.

HRMS (ESI): Calculated for C₁₄H₁₁N₃O₈SNa [M+Na]+: 404.0159. Found 404.0140.

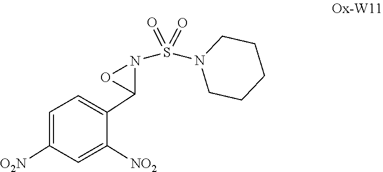 [SMILES: O=[N+]([O-])c1ccc(C2ON2S(=O)(=O)N2CCCCC2)c([N+](=O)[O-])c1]

Ox-W11 was prepared according to the standard procedure. The crude material was initially purified on a silica gel Biotage column (25G) with a gradient of 10-30% EtOAc/hexanes, but remained partially impure by TLC analysis. A portion of this material was dissolved in CH₂Cl₂, loaded onto and purified on a preparatory plate (silica gel base) using 30% EtOAc/hexanes as the mobile phase. A yellow oil which crystallized to form a yellow-white solid (176 mg, 0.49 mmol, 16% yield from 3 mmol of sulfonamide starting material) was obtained upon storage at −20° C.

¹H NMR (500 MHz, Chloroform-d) δ 9.07 (d, J=2.3 Hz, 1H), 8.57 (dd, J=8.5, 2.3 Hz, 1H), 7.90 (d, J=8.5 Hz, 1H), 6.05 (s, 1H), 3.68-3.35 (m, 4H), 1.78-1.69 (m, 4H), 1.68-1.61 (m, 2H).

¹³C NMR (126 MHz, CDCl₃) δ 149.1, 148.5, 134.5, 131.0, 128.6, 120.6, 74.1, 48.2, 25.5, 23.6.

HRMS Calculated for C₁₂H₁₄N₄O₇SNa [M+Na]⁺=381.0475. Measured [M+Na]⁺=381.0486.

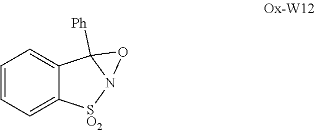 [SMILES: O=S1(=O)c2ccccc2C2(c3ccccc3)ON21]

Synthesized according to a reported literature procedure.⁸ Spectral data matched literature reports.

¹H NMR (400 MHz, CDCl₃) δ 7.92-7.85 (m, 1H), 7.75 (m, 2H), 7.67-7.47 (m, 6H).

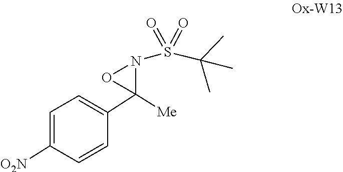 [SMILES: CC1(c2ccc([N+](=O)[O-])cc2)ON1S(=O)(=O)C(C)(C)C]

Adapted from a literature procedure.⁹ To a solution of 2-methylpropane-2-sulfinamide (485 mg, 4 mmol, 1.0 equiv.) and 4-nitroacetophenone (793 mg, 4.8 mmol, 1.2 equiv.) in dry THF (13 mL) was added Ti(iPrO)₄ (2.4 mL, 8 mmol, 2 equiv.) dropwise at room temperature. The reaction was sealed and heated at 70° C. for 48 h. Water (10 mL) was added and the mixture was filtered washing with CH₂Cl₂. The filtrate was extracted with CH₂Cl₂, then the combined organic layers was washed with brine, dried over Na₂SO₄, filtered and concentrated in vacuo. Purification by column chromatography afforded the desired imine (775 mg, 72%) as a dark red oil.

Dry mCPBA (˜85%, 223 mg, 1.1 mmol, 1.1 equiv.) was taken up in CH₂Cl₂ (2.5 mL), followed by the addition of KOH (powdered, 196 mg, 3.5 mmol, 3.5 equiv.) to form a white suspension which was stirred at room temperature 5 minutes. In a separate vial, the imine from the previous step (268 mg, 1 mmol, 1 equiv.) was dissolved in CH₂Cl₂ (5 mL), followed by the addition of dry mCPBA (˜85%, 203 mg, 1 mmol, 1 equiv.). The reaction was stirred at room temperature 1 minute, and then added to the initial stirring basic white mCPBA suspension.

The reaction was monitored by 1H NMR using small aliquots dissolved in C₆D₆. After 16-48 h, the reaction mixture was diluted with CH₂Cl₂ (50 mL) and water (50 mL), and extracted with additional CH₂Cl₂ (3×50 mL). The combined organic phases were concentrated in vacuo until a total volume of ˜40 mL to afford a solution of crude imine which was used directly in the next step. After 5 minutes, the reaction was concentrated in vacuo to afford a light yellow oil. Trituration with 2:1 Et₂O/pentane afforded the desired oxaziridine (300 mg, >99%) as an off-white solid (2.5:1 d.r.).

¹H NMR (300 MHz, CDCl₃) Major diastereomer: δ 8.25 (m, 2H, major), 7.69-7.62 (m, 2H), 2.40 (s, 3H), 1.56 (s, 9H). Minor diastereomer: δ 8.25 (m, 2H), 7.74 (m, 2H), 7.69-7.62 (m, 2H), 1.93 (s, 3H), 1.49 (s, 9H).

¹³C NMR (126 MHz, CDCl₃) major diastereomer: δ 148.8, 143.9, 127.7, 123.9, 82.7, 62.5, 24.0, 17.5.

HRMS (ESI): Calculated for C₁₂H₁₆N₂O₅SNa [M+Na]+: 323.0672. Found 323.0653.

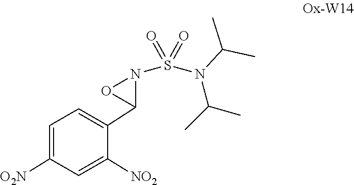 [SMILES: CC(C)N(C(C)C)S(=O)(=O)N1OC1c1ccc([N+](=O)[O-])cc1[N+](=O)[O-]]

General Procedure A was followed starting from diisopropylamine-1-sulfonamide (110 mg, 0.76 mmol, 1.0 equiv.) and 2,4-dinitrobenzaldehyde (179 mg, 0.91 mmol, 1.2 equiv.). Purification by column chromatography (0% to 30% EtOAc/hexane) afforded the corresponding oxaziridine (38 mg, 15%) as a white solid.

¹H NMR (300 MHz, CDCl₃) δ 9.06 (d, J=2.3 Hz, 1H), 8.55 (dd, J=8.5, 2.3 Hz, 1H), 7.90 (d, J=8.5 Hz, 1H), 6.07 (s, 1H), 3.94 (hept, J=6.9 Hz, 2H), 1.38 (m, 12H).

¹³C NMR (151 MHz, CDCl₃) δ 149.0, 148.7, 134.8, 131.2, 128.5, 120.5, 74.4, 50.6, 22.4, 21.9.

HRMS (ESI): Calculated for C₁₃H₁₈N₄O₇SNa [M+Na]+: 397.0788. Found 397.0763.

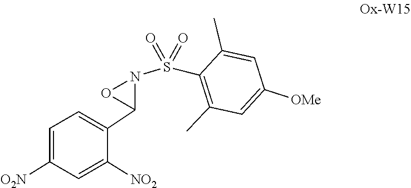 [SMILES: COc1cc(C)c(S(=O)(=O)N2OC2c2ccc([N+](=O)[O-])cc2[N+](=O)[O-])c(C)c1]

General Procedure A was followed starting from 4-methoxy-2,6-dimethylbenzenesulfonamide (646 mg, 3 mmol, 1.0 equiv.) and 2,4-dinitrobenzaldehyde (706 mg, 3.6 mmol, 1.2 equiv.). Purification by column chromatography (10% to 50% EtOAc/hexane) afforded the corresponding oxaziridine (218 mg, 18%) as a white solid. ¹H NMR (300 MHz, CDCl₃) δ 9.07 (d, J=2.2 Hz, 1H), 8.55 (dd, J=8.5, 2.2 Hz, 1H), 7.86 (d, J=8.6 Hz, 1H), 6.72 (s, 2H), 6.25 (s, 1H), 3.86 (s, 3H), 2.75 (s, 6H). ¹³C NMR (126 MHz, CDCl₃) δ 163.5, 149.1, 148.5, 145.3, 134.4, 131.1, 128.6, 123.2, 120.6, 116.7, 73.5, 55.6, 23.9. HRMS (ESI): Calculated for C₁₆H₁₉N₄O₈S [M+NH₄]+: 427.0918. Found 427.0916.

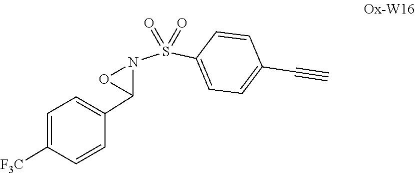 [SMILES: C#Cc1ccc(S(=O)(=O)N2OC2c2ccc(C(F)(F)F)cc2)cc1]

General Procedure A was followed starting from 4-ethynylbenzenesulfonamide (91 mg, 0.5 mmol, 1.0 equiv.) and 4-trifluoromethylbenzaldehyde (0.082 mL, 0.6 mmol, 1.2 equiv.). Purification by column chromatography (0% to 20% EtOAc/hexane) afforded the corresponding oxaziridine (72 mg, 41%) as a beige solid. ¹H NMR (300 MHz, CDCl₃) δ 8.01 (d, J=8.4 Hz, 2H), 7.78-7.64 (m, 4H), 7.58 (d, J=8.2 Hz, 2H), 5.56 (s, 1H), 3.36 (s, 1H). ¹³C NMR (151 MHz, CDCl₃) δ 134.4, 133.6 (q, J=32.8 Hz), 133.1, 129.7, 129.5, 128.9 (2C), 125.9 (q, J=3.7 Hz), 123.6 (q, J=273 Hz) 82.4, 81.7, 75.4. ¹⁹F NMR (376 MHz, CDCl₃) δ −62.2.

HRMS (ESI): Calculated for C₁₆H₁₄F₃N₂O₃S [M+NH₄]+: 371.0671. Found 371.0659.

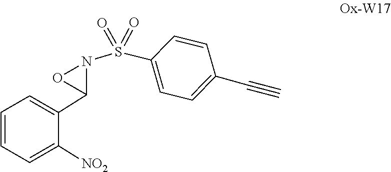 [SMILES: C#Cc1ccc(S(=O)(=O)N2OC2c2ccccc2[N+](=O)[O-])cc1]

General Procedure A was followed starting from 4-ethynylbenzenesulfonamide (91 mg, 0.5 mmol, 1.0 equiv.) and 2-nitrobenzaldehyde (91 mg, 0.6 mmol, 1.2 equiv.). Purification by column chromatography (10% to 50% EtOAc/hexane) afforded the corresponding oxaziridine (48 mg, 29%) as a white solid. ¹H NMR (300 MHz, CDCl₃) δ 8.25 (d, J=8.0 Hz, 1H), 8.05 (d, J=8.5 Hz, 2H), 7.74-7.69 (m, 3H), 7.65 (m, 1H), 7.60 (m, 1H), 6.06 (s, 1H), 3.35 (s, 1H).

¹³C NMR (126 MHz, CDCl₃) δ 148.0, 134.8, 133.5, 133.0, 131.6, 130.0, 129.6, 129.0, 127.7, 125.3, 82.4, 81.8, 75.4. HRMS (ESI): Calculated for C₁₅H₁₀N₂O₅SNa [M+Na]+: 353.0202. Found 353.0181.

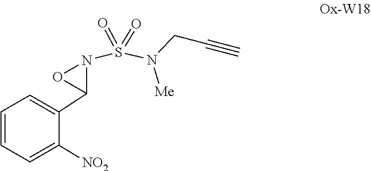 [SMILES: C#CCN(C)S(=O)(=O)N1OC1c1ccccc1[N+](=O)[O-]]

General Procedure A was followed starting from the corresponding sulfonamide (74 mg, 0.5 mmol, 1.0 equiv.) and 2-nitrobenzaldehyde (91 mg, 0.6 mmol, 1.2 equiv.). Purification by column chromatography (10% to 30% EtOAc/hexane) afforded the corresponding oxaziridine (101 mg, 68%) as a white solid. ¹H NMR (500 MHz, CDCl₃) δ 8.29-8.23 (m, 1H), 7.75 (td, J=7.6, 1.3 Hz, 1H), 7.70-7.62 (m, 2H), 6.02 (s, 1H), 4.25-4.17 (m, 2H), 3.19 (s, 3H), 2.39 (t, J=2.5 Hz, 1H). ¹³C NMR (126 MHz, CDCl₃) δ 148.2, 134.7, 131.4, 129.1, 128.1, 125.2, 76.7, 75.2, 74.6, 40.9, 35.6. HRMS (ESI): Calculated for C₁₁H₁₅N₄O₅S [M+NH₄]+: 315.0757. Found 315.0763.

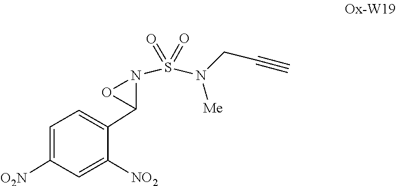 [SMILES: C#CCN(C)S(=O)(=O)N1OC1c1ccc([N+](=O)[O-])cc1[N+](=O)[O-]]

General Procedure A was followed starting from the corresponding sulfonamide (74 mg, 0.5 mmol, 1.0 equiv.) and 2,4-dinitrobenzaldehyde (118 mg, 0.6 mmol, 1.2 equiv.). Purification by column chromatography (5% to 30% EtOAc/hexane) afforded the corresponding oxaziridine (75 mg, 44%) as a white solid.

¹H NMR (500 MHz, CDCl₃) δ 9.08 (d, J=2.2 Hz, 1H), 8.57 (dd, J=8.5, 2.3 Hz, 1H), 7.90 (d, J=8.5 Hz, 1H), 6.09 (s, 1H), 4.23 (t, J=2.4 Hz, 2H), 3.19 (s, 3H), 2.41 (t, J=2.5 Hz, 1H). ¹³C NMR (151 MHz, CDCl₃) δ 149.2, 148.5, 134.1, 131.0, 128.7, 120.6, 76.4, 74.8, 74.3, 40.9, 35.6. HRMS (ESI): Calculated for C₁₁H₁₀N₄O₇SNa [M+Na]+: 365.0162. Found 365.0137.

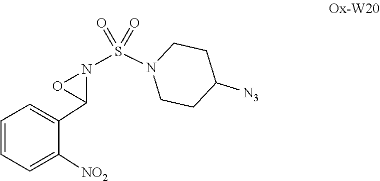 [SMILES: [N-]=[N+]=NC1CCN(S(=O)(=O)N2OC2c2ccccc2[N+](=O)[O-])CC1]

General Procedure A was followed starting from the corresponding sulfonamide (103 mg, 0.50 mmol, 1.0 equiv.) and 2-nitrobenzaldehyde (91 mg, 0.60 mmol, 1.2 equiv.). Purification by column chromatography (20% to 80% EtOAc/hexane, then 70% to 100% DCM/hexane) afforded the corresponding oxaziridine (110 mg, 62%) as a colorless oil.

¹H NMR (500 MHz, Chloroform-d) δ 8.25 (dd, J=8.0, 1.3 Hz, 1H), 7.76 (td, J=7.5, 1.3 Hz, 1H), 7.70-7.60 (m, 2H), 6.00 (s, 1H), 4.01-3.60 (m, 3H), 3.55-3.43 (m, 2H), 2.16-1.92 (m, 2H), 1.87-1.72 (m, 2H).

¹³C NMR (126 MHz, CDCl₃) δ 148.1, 134.7, 131.4, 129.0, 128.1, 125.2, 75.4, 56.2, 44.4, 44.4, 30.2, 30.2. HRMS Calculated for C₁₂H₁₄N₆O₅SNa [M+Na]+=377.0639. Found [M+Na]+=377.0611.

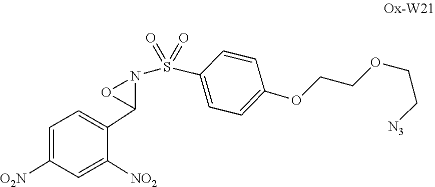 [SMILES: [N-]=[N+]=NCCOCCOc1ccc(S(=O)(=O)N2OC2c2ccc([N+](=O)[O-])cc2[N+](=O)[O-])cc1]

General Procedure A was followed starting from the corresponding sulfonamide (44 mg, 0.16 mmol, 1.0 equiv.) and 2,4-dinitrobenzaldehyde (37 mg, 0.19 mmol, 1.2 equiv.). Purification by column chromatography (20% to 80% EtOAc/hexane) afforded the corresponding oxaziridine (27 mg, 36%) as a white solid.

¹H NMR (500 MHz, CDCl₃) δ 9.05 (d, J=2.2 Hz, 1H), 8.53 (dd, J=8.5, 2.2 Hz, 1H), 8.04-7.97 (m, 2H), 7.84 (d, J=8.5 Hz, 1H), 7.15-7.09 (m, 2H), 6.04 (s, 1H), 4.28-4.23 (m, 2H), 3.94-3.88 (m, 2H), 3.75 (m, 2H), 3.41 (m, 2H). ¹³C NMR (126 MHz, CDCl₃) δ 164.7, 149.2, 148.4, 134.0, 132.6, 131.0, 128.7, 124.2, 120.6, 115.5, 74.4, 70.6, 69.5, 68.2, 50.8. HRMS (ESI): Calculated for C₁₇H₂₀N₇O₉S [M+NH₄]+: 498.1037. Found 498.1020.

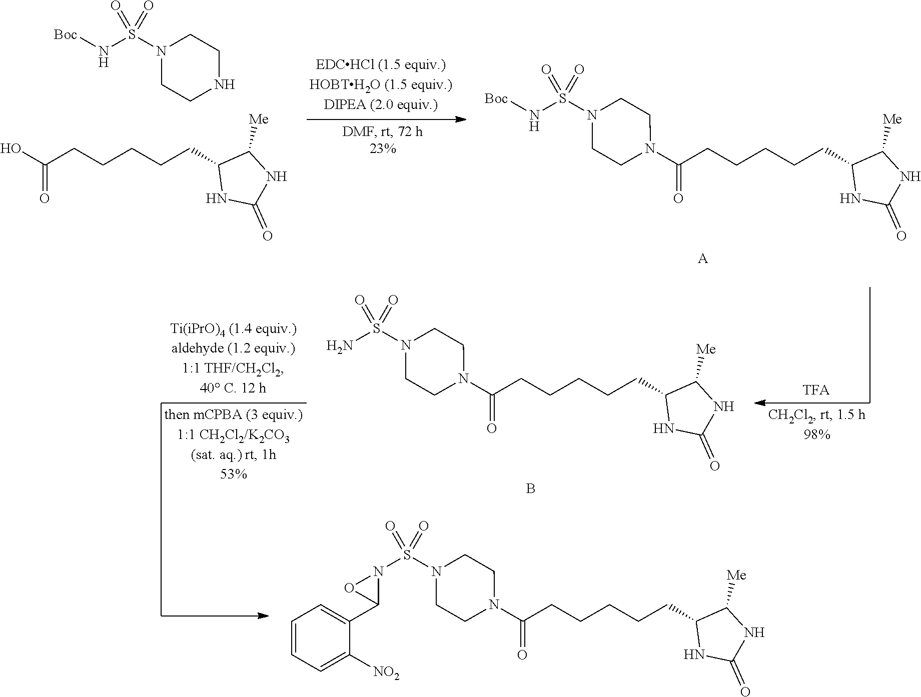

To a solution of tert-butyl (piperazin-1-ylsulfonyl)carbamate (275 mg, 1.04 mmol, 1.0 equiv.) in DMF (10 mL) was added desthiobiotin (223 mg, 1.04 mmol, 1.0 equiv.), EDC·HCl (307 mg, 1.6 mmol, 1.5 equiv.), HOBT-H₂O (245 mg, 1.6 mmol, 1.5 equiv.), and DIPEA (0.37 mL, 2.1 mmol, 2.0 equiv.). After 72 h stirring at room temperature, the crude reaction mixture was directly subjected to column chromatography (6% to 10% MeOH/CH₂Cl₂) to afford the desired product A (110 mg, 23%) as a white solid containing a small HOBT impurity (˜10%).

Compound A (100 mg, 0.22 mmol, 1.0 equiv.) was dissolved in CH₂Cl₂ (3.6 mL) and TFA (0.9 mL). After stirring at room temperature for 1.5 h, the solvent was removed in vacuo. Purification by column chromatography (10% MeOH/CH₂Cl₂) afforded the corresponding sulfonamide B (76 mg, 98%) as a white solid.

To a solution of sulfonamide B (52 mg, 0.14 mmol, 1.0 equiv.) and 2-nitrobenzaldehyde (25 mg, 0.17 mmol, 1.2 equiv.) in dry THF (0.7 mL) and CH₂Cl₂ (0.7 mL) was added Ti(iPrO)₄ (59 L, 0.20 mmol, 1.4 equiv.) dropwise at room temperature. The mixture was heated to 40° C. for 12 h. The solvent was removed in vacuo to afford crude imine which was used directly in the next step.

In a 25 mL rbf, mCPBA (75%, 50 mg, 0.38 mmol, 5.4 equiv.) was prestirred in 1:1 CH₂Cl₂/sat. aq. K₂CO₃ (2 mL) at room temperature for 10 minutes. The solution of crude imine from the previous step (in 1 mL CH₂Cl₂) was added dropwise, using additional CH₂Cl₂ (1 mL) rinses for a quantitative transfer. After 2.5 h, the reaction was diluted with CH₂Cl₂ (50 mL) and sat. aq. NaHCO₃ (15 mL), and extracted with CH₂Cl₂ (2×30 mL). The combined organic layers were washed with brine (30 mL), dried over Na₂SO₄, filtered and concentrated in vacuo. Purification by column chromatography (10% MeOH/CH₂Cl₂) afforded the oxaziridine linked to Desthiobiotin (38 mg, 53%) as a white solid.

¹H NMR (600 MHz, CDCl₃) δ 8.25 (dd, J=8.2, 1.2 Hz, 1H), 7.75 (td, J=7.6, 1.3 Hz, 1H), 7.66 (td, J=7.8, 1.5 Hz, 1H), 7.62 (dd, J=7.7, 1.5 Hz, 1H), 6.01 (s, 1H), 5.30 (s, 1H), 4.87 (s, 1H), 3.82 (m, 1H), 3.78-3.70 (m, 2H), 3.67 (m, 1H), 3.64-3.49 (m, 6H), 2.34 (t, J=7.5 Hz, 2H), 1.64 (p, J=7.3 Hz, 2H), 1.54-1.22 (m, 6H), 1.10 (d, J=6.4 Hz, 3H). ¹³C NMR (126 MHz, CDCl₃) δ 171.7, 163.7, 148.0, 134.7, 131.5, 128.9, 127.9, 125.2, 75.5, 56.1, 51.5, 47.1, 47.0, 45.3, 41.2, 33.0, 29.6, 29.3, 26.3, 24.9, 15.8. HRMS (ESI): Calculated for C₂₁H₃₁N₆O₇S [M+H]+: 511.1970. Found 511.1956.

### 2. Selectivity and Reactivity of Ac-Trp-OMe with N-Sulfonyl Oxaziridines

### General Procedure

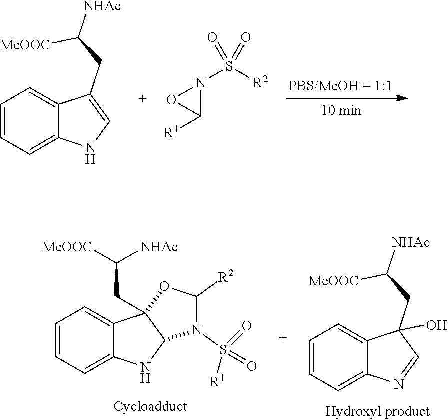

A stock solution of Ac-Trp-OMe (100 mM) in MeOH was mixed with oxaziridine (100 mM) in MeOH (v/v ratio=1:1.2), followed by the immediate addition of PBS (1 volume equiv. to make 1:1 PBS/MeOH). For some oxaziridines with low solubility, the reaction was performed in PBS/MeOH/MeCN (1:1:1) co-solvent. The reaction was stirred for 10 min at room temperature, and then analyzed by LC-MS. It is important the initial stock solutions of Ac-Trp-OMe and oxaziridine are fully homogeneous prior to mixing.

For LC-MS analysis, solvent A was water and solvent B was methanol. The linear gradient was 40-100% B in 6.5 min and 100% B for 1.5 min.

### 3. Molecular Orbital Calculations

Molecular orbital calculations for model oxaziridines were computed using the Spartan '20 program from Wavefunction, Inc. Molecules were built and their geometries minimized using the Spartan 20' GUI and submitted to an ‘Equilibrium Geometry’ calculation at ‘Ground’ state in ‘Water’ with ‘Density Functional wB97X-D’ method and a ‘6-31G*’ basis set.

### 4. Characterization of the Spectroscopic and Kinetics of the Trp-CLiC Reactions

The absorbance of unlabeled Ac-Trp-OMe and purified cycloadduct in PBS buffer were detected by UV-Vis equipment. To gain the A260/A280 ratio against the Trp/Ox ratio, different concentrations of Ox-W18 from 0 to 100 μM was added to the Ac-Trp-OMe (100 μM) in PBS buffer at 25° C., and then the absorbance was detected after 20 min for reaction completion.

For kinetics study of the Trp-CLiC reaction, the absorbance at 280 nm was time-dependently monitored by UV-Vis upon adding Ox-W18 (50 μM) into different concentrations of Ac-Trp-OMe (500 μM, 550 μM and 600 μM) in PBS buffer at 25° C. Then the second-order reaction rate constant could be obtained.

### 5. Oxaziridine Screening on Peptides

Peptides were dissolved in 5% DMSO solution. Different N-sulfonyl oxaziridines (100 mM stock in DMF) were added to 50 μl peptide solution (50 μM) and the final concentration was 250 μM. The reactions were performed for 10 min at room temperature and then quenched by addition of excess Ac-Met-OMe solution. The labeled peptides were subjected to Q-TOF analysis with a Proswift RP-4H (monolithic phenyl, 1.0 mm×50 mm, Dionex) column. The Solvent A was MilliQ water with 0.1% (v/v) formic acid, while the Solvent B was ACN with 0.1% (v/v) formic acid. The LC system was set at the 0.30 mL/min with a gradient of 5-100% solvent B in solvent A over 8 min. MassHunter Quantitative Analysis Version software was used for ion chromatogram extraction and Maximum Entropy deconvolution.

### 6. Protein Labelling with N-Sulfonyl Oxaziridine

Proteins were dissolved in Tris (50 mM, pH 8.0) buffer. Ox-W18 (100 mM stock in DMF) was added to 30 μL protein solution (50 μM) and the final concentration was 300 μM. The reactions were performed for 10 min at room temperature and then quenched by addition of excess Ac-Met-OMe solution. For protein labelling under denaturing condition, the protein solution was treated with 0.5% SDS and then boiled for 5 min at 95° C. before Ox-W18 addition. For competition assay, free Ac-Trp-OMe (2 mM) was added beforehand. For protein labelling under different concentrations of GdmCl, high-concentration Lysozyme was diluted into GdmCl buffers and final protein concentration was set as 50 μM. Lysozyme protein would be stored in GdmCl buffers for three hours to form stable conformations before labelling with Ox-W18. After tryptophan labelling with Ox-W18, the proteins were desalted with Zeba™ Spin Desalting Columns (7K MWCO, 0.5 mL) before subsequent Q-TOF analysis and UV-Vis analysis or further click reaction. In-gel fluorescence imaging was also applied to test the tryptophan labelling efficiency. For the copper-mediated alkyne-azide cyclization reaction of fluorescence dye, 100 μM CuSO₄, 100 μM TBTA, 100 μM azide-dye and 2 mM sodium ascorbate was added to Ox-W18-labeled proteins. This reaction was performed at room temperature for 1 h.

### 7. Chemoselective Tryptophan Profiling

The desired proteome sample was diluted to a 2.5 mg/mL solution. Then Ox-W18 probe was added to each sample, typically 1 mM as the final concentration. After incubation at room temperature for half one hour, this reaction was quenched by excess Ac-Met-OMe. For click reaction, 40 μl 100 mM sodium ascorbate, 8 μl 50 mM DADPS azide-biotin in DMSO and 200 ul Cu-TBTA mixture [200 ul TBTA ligand solution (0.9 mg/mL in DMSO:t-butanol=1:4)+20 ul 100 mM CuSO₄] were added to 1.8 mL sample solution and then incubated for one hour. The proteome was precipitated by MeOH/CHCl₃ (4:1.5). After washed with pre-chilled cold methanol for three times, the pellet was re-dissolved in 1 mL 0.2% SDS/PBS (w/v). Biotin labeled proteome was enriched with pre-washed high-capacity agarose beads on a rotator at 4° C. overnight. Then on-beads digestion was performed. 800 μL fresh 6M Urea/PBS was added to the beads (total volume is about 1 ml), and then the proteome reduction (5 mM DTT) and IA alkylation (10 mM IAA) were done in order. Then the buffer was changed to 1M Urea/PBS (with 0.1% SDS, 1 mM CaCl₂). After addition of 8 μL of 0.5 μg/μl Trypsin solution, the mixture was Incubated on a rotator at 37° C. for 16-18 h. Then the beads were washed very carefully followed by acid-cleavage in 5% formic acid in water to afford the probed tryptophan-containing peptides. Liquid was removed using SpeedVac and lyophilizer. Before mass spectrometric analysis, peptides were reconstituted in 200 μL 0.1% FA solution.

### 8. In-Solution Digestion of IL8, Lysozyme, and Proteome Samples

The precipitated protein or proteome were re-dissolved in the dissolving buffer (PBS, 6 M urea, pH 7.5). 100 mM DTT was added to a final concentration of 5 mM, and then reacted for 30 min at 37° C. 1 M IAA was added to a final concentration of 15 mM, and then reacted for 30 min at room temperature and in the dark. After reduction and alkylation, Trypsin was added at a 50:1 protein:protease ratio (w/w), then mixed and incubated for 18 hours at 37° C. The in-solution digestion was terminated by adding trifluoroacetic acid to a final concentration of 0.5-1%. Particulate material was removed by centrifuging at 16,000 g for 10 min. The collected peptides could be examined by LC-MS/MS.

### 9. LC-MS/MS Analysis

Trypsin digested peptides were analyzed on a Thermo Scientific Q Exactive™ Plus Hybrid Quadrupole-Orbitrap™ Mass Spectrometer coupled with an Aligent 1260 Infinite LC system. The mobile phases were A: 0.1% formic acid in 95% H₂O-20% ACN; B: 0.1% formic acid in 80% ACN-20% H₂O. The MS/MS analysis was performed under the positive-ion mode with a full-scan m/z range from 400 to 1,800 and a mass resolution of 70,000. Peptides were eluted using a 90 min gradient (0-5 min 100% A; 60 min 50% A; 70 min 100% A; 70-90 min 100% A) with a flow of 0.1 mL/min. MS/MS fragmentation was performed in a data-dependent mode, of which TOP 15 most intense ions are selected for MS2 analysis with a resolution of 17,500 using the collision mode of CID. Other important parameters: isolation window, 1.6 m/z units; default charge, +2, charge exclusion, unassigned, 8, >8; normalized collision energy, 27%; maximum IT, 50 ms; dynamic exclusion, 60.0 s.

### 10. Peptide Identification in Proteomics Experiments

For protein ID and modification identification, all MS/MS raw files were analyzed using pFind platform. Alkylation of cysteine (+57.0215 Da) was set as the fixed modification, and oxidation of methionine and tryptophan (+15.9949 Da) was assigned as the variable modifications. Besides, for MS/MS analysis of IL8 or Lysozyme, tryptophan oxidative cycloadduct was also seen as the modification (+297.0419 for tryptophan cycloadduct and −281.0470 Da for neutral loss); for ABPP analysis, product after acid-cleavage was regarded as the final modification (+440.1478 for tryptophan cycloadduct and −424.1529 Da for neutral loss). This software was set up to search peptides with precursor mass accuracy at ±20 ppm, fragment ion mass accuracy at ±20 ppm, and the results were filtered by applying a 1% FDR.

### 11. Preparation and Characterization of Her2-Fab Antibodies

All tryptophan mutants were made using QuikChange to introduce single codon mutations onto Fab. Fabs were expressed and purified by an optimized autoinduction protocol previously described.¹⁰ In brief, C43 (DE3) Pro+ E. coli containing expression plasmids were grown in TB autoinduction media at 37° C. for 6 h, then cooled to 30° C. for 16 to 18 h. The bacterial cells were spun down and lysed with B-PER detergent, followed by incubation at 60° C. for 20 minutes. Following a hard spin at 30,000 g for 20 minutes, the Fabs in the lysed supernatant were purified by Protein A affinity chromatography. Fab purity and integrity was assessed by SDS-PAGE and intact protein mass spectrometry using a Xevo G2-XS Mass Spectrometer (Waters) equipped with a LockSpray (ESI) source and Acquity Protein BEH C4 column (2.1 mm inner diameter, 50 mm length, 300 Å pore size, 1.7 μm particle size) connected to an Acquity I-class liquid chromatography system (Waters). Deconvolution of mass spectra was performed using the maximum entropy (MaxEnt) algorithm in MassLynx 4.1 (Waters).

### 12. Her2-Fab Labeling with Ox-W18 and Stability Studies

The Fab antibodies were incubated at 50 μM with 3 molar equivalents of the Ox-W18 (150 μM). After labeling for 2 hours, the Fabs were buffer exchanged into PBS using a 0.5-mL Zeba 7-kDa desalting column (Thermo Fisher Scientific). Conjugation was monitored by intact protein mass spectrometry using a Xevo G2-XS Mass Spectrometer (Waters). For the stability studies, the Fabs were labeled as stated above and placed in an incubator at 37° C. 5 mL of the sample were taken at the indicated timepoints, flash frozen, and stored at −20° C. All time points were measured by intact protein mass spectrometry.

### 13. Unfolding Stress Detection of Diverse Inhibitors in Living Cells

HeLa cells were treated with MG-132 (5 μg/ml) or Tunicamycin (5 μM) for 8 hours. Then cells were harvested and lysed in PBS buffer (1% Triton, with protease inhibitors). The proteome concentration was adjusted to 1 mg/ml and N-sulfonyl oxaziridine with desthiobiotin (250 μM) was added for proteome labeling. Reactions were quenched by excess Ac-Met-OMe solution. The labeling results were detected by Western blot analysis against biotin.

### 14. Immunofluorescence Imaging

HEK 293T cells in 8-well chamber slides were cultured in DMEM contained with 10% FBS. NPM1 and NONO WT and mutant plasmids were transfected at the point of 50% confluency by Lipofectamine 2000 Transfection Reagent. After 6 h, the cells were changed to fresh DMEM (10% FBS) medium. Then cells were Hoechst stained after 24 h expression for 20 min. The immunofluorescence imaging processes were performed using a Zeiss LSM700 laser scanning confocal microscope with a 63× oil-immersion objective lens using Zen 2009 software (Carl Zeiss). Image analysis and quantification was performed using Fiji.

### 15. NMR Spectra

See priority application.

### SUPPLEMENTAL REFERENCES

1. F. A. Davis et al, J. Am. Chem. Soc. 102, 2000-2005, (1980).
2. A. Ghosh et al, Org. Lett. 18, 4940-4943, (2016).
3. S. Dong et al, J. Am. Chem. Soc. 135, 10026-10029, (2013).
4. X. Gu, Y. Zhang, Z.-J. Xu, C.-M. Che, Chem. Commun. 50, 7870-7873, (2014).
5. L. Lykke, C. Rodriguez-Escrich, K. A. Jorgensen, J. Am. Chem. Soc. 133, 14932-14935, (2011).
6. W. B. Jennings, S. P. Watson, D. R. Boyd, J. Chem. Soc., Chem. Commun. 14, 931-932, (1988).
7. R. W. F. Kerr et al, Tetrahedron Asymmetry 28, 125-134. 2017
8. B. Song et al, Org. Lett. 17, 190-193, (2015).
9. J. L. G. Ruano, J. Aleman, C. Fjardo, A. Parra, Org. Lett. 7, 5493-5496, (2005).
10. S. K. Elledge et al. Proc. Natl. Acad. Sci. U.S.A 117, 5733-5740, (2020).

1. A method of chemoselective conjugation comprising reacting an N-sulfonyl oxaziridine with an indole substrate in an oxidative cyclization reaction in an aqueous, biocompatible environment under conditions to form a resultant cycloadduct conjugation product.

2. The method of claim 1 wherein the N-sulfonyl oxaziridine is of formula I, the indole substrate is of formula II, and the cycloadduct is of the corresponding formula III: 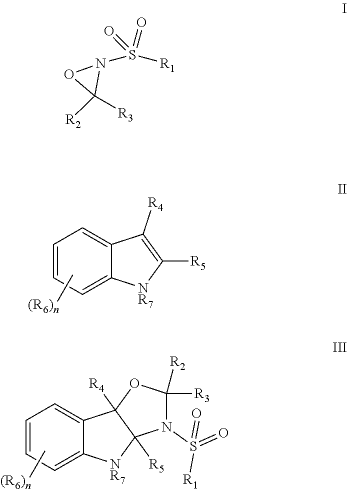
wherein R1-R7 are independently selected from optionally substituted heteroatom and optionally substituted, optionally hetero-, optionally cyclic C1-C18 hydrocarbyl, and n is an integer 1-5, preferably 1-3 or 1-2.

3. The method of claim 2 wherein:
R1-R3 and R5-R7 are independently H, C1-C4 alkyl (Me, Et, Pr, Bu) or fully or partially fluorinated C1-C4 alkyl (e.g. CF₃), sulfanyl or fluorosulfanyl (e.g. SFs), C1-C4 alkoxy/ether, ester or carboalkoxy (e.g. OMe, OOMe, or CO₂Me), CN, NO₂, or phenyl or substituted phenyl, with n substituents, preferably selected from C1-C4 alkyl (Me, Et, Pr, Bu), fully or partially fluorinated C1-C4 alkyl (e.g. CF₃), sulfanyl or fluorosulfanyl (e.g. SFs), C1-C4 alkoxy/ether, ester or carboalkoxy (e.g. OMe, OOMe, or CO₂Me), CN, NO₂.

4. The method of claim 2, wherein
R4 is an alpha carbon of an amino acid, preferably tryptophan, wherein the amine of the amino acid may be acetylated and the carboxyl may be O-methylated, and wherein the amino acid may be a residue of a protein.

5. The method of claim 2, wherein:
R4 is a residue of a protein.

6. The method of claim 2, wherein:
R2 is substituted or unsubstituted phenyl;
R3 is H;
R5 is H;
R6 is H;
R7 is H; or
R8 is H.

7. The method of claim 2, wherein:
R2 is substituted or unsubstituted phenyl;
R3 is H;
R4 is an alpha carbon of an amino acid, preferably tryptophan, wherein the amine of the amino acid may be acetylated and the carboxyl may be O-methylated, and wherein the amino acid may be a residue of a protein;
R5 is H;
R6 is H;
R7 is H; and
R8 is H.

8. The method of claim 1, wherein the indole substrate is a tryptophan substrate, and the method provides a residue-specific bioconjugation strategy for tryptophan-based substrate functionalization.

9. The method of claim 1, wherein the indole substrate is a tryptophan substrate of a peptide, a polypeptide, or a protein and the method results in site- and residue-specific modification of the protein, with applications in synthesis and characterization of antibody-drug conjugates and related biologic therapeutics and imaging agents, chemoproteomics and inhibitor design, as well as modifications to study and improve upon protein function, including solubility, stability, and metabolism and pharmacokinetics.

10. The method of claim 1, combined with stable isotope labeling with amino acid in cell culture (SILAC) or isotope coded affinity tag (ICAT) for quantitative proteomics analysis of tryptophan function in vivo and in vitro by mass spectrometry, with application including but not limited to quantitative analysis of tryptophan reactivity, quantitative analysis of oxidative-sensitive tryptophan, quantitative analysis of C-mannosylated tryptophan and quantitative analysis of C-mannosyltransferase DPY19 substrates.

11. A composition comprising an N-sulfonyl oxaziridine of formula I.

12. The composition of claim 11, comprising a mixture of the N-sulfonyl oxaziridine, and an indole substrate of formula II.

13. The composition of claim 11, comprising a mixture of the N-sulfonyl oxaziridine, an indole substrate of formula II, and a cycloadduct of the corresponding formula III.

14. The composition of claim 11, wherein the indole substrate is a tryptophan substrate, preferably a tryptophan substrate of a protein.

15. The composition of claim 11, herein in an aqueous, biocompatible medium, particularly wherein the indole substrate is a tryptophan substrate, preferably a tryptophan substrate of a protein.
